(19) United States
(12) Patent Application Publication
(10) Pub. No.: US 2008/0227096 A1
(43) Pub. Date: Sep. 18, 2008

(54) Assay for response to proteasome inhibitors

(75) Inventors: Silvia Chiu Wah Ling, Sydney, N.S.W. (AU); John David Allen, Jannali, N.S.W. (AU)

Correspondence Address: ROTHWELL, FIGG, ERNST & MANBECK, P.C., 1425 K STREET, N.W., SUITE 800, WASHINGTON, DC, 20005, US

(73) Assignee: Centenary Institute of Cancer Medicine and Cell Biology, Camperdown, N.S.W. (AU)

(21) Appl. No.: 12/000,091

(22) Filed: Dec. 7, 2007

Related U.S. Application Data
(60) Provisional application No. 60/960,760, filed on Oct. 12, 2007.

(30) Foreign Application Priority Data
Dec. 8, 2006 (AU) 2006906900
Sep. 5, 2007 (AU) 2007904810
Oct. 12, 2007 (AU) 2007221966

(51) Int. Cl. C12Q 1/68

(52) U.S. Cl. 435/6

(57) ABSTRACT

The invention relates to a method for predicting a response to a proteasome inhibitor in the prophylaxis or treatment of a cancer in an individual. The method comprises providing a sample of cancer cells of the cancer from the individual, and evaluating the level of at least one molecule in the cancer cells associated with the unfolded protein response of the cancer cells, to provide test data indicative of the level of activity of the unfolded protein response. The test data is used to predict the response of the cancer cells to the proteasome inhibitor. The evaluation of the level of the molecule can be utilized for determination of treatment for the cancer.

### CROSS-REFERENCE TO RELATED APPLICATIONS

The present invention is related to and claims priority under 35 U.S.C. §119(e) to U.S. Provisional Patent Application Ser. No. 60/960,760 filed 12 Oct. 2007. This application is incorporated herein by reference. The present application also claims priority under 35 U.S.C. §119(d) to Australian Provisional Application No. 2006906900 filed 8 Dec. 2006, Australian Provisional Application No. 2007904810 filed 5 Sep. 2007 and Australian Complete Application No. 2007221966 filed 12 Oct. 2007.

### FIELD OF THE INVENTION

The invention relates to a method for assessing resistance or sensitivity of cancer cells to a proteasome inhibitor. The assessment provides data which finds application for the prediction of response of the cancer cells to therapeutic treatment with the proteasome inhibitor and evaluation of the prognosis of the cancer.

### BACKGROUND OF THE INVENTION

Multiple myeloma is a malignant proliferation of plasma cells in the bone marrow that remains incurable by chemotherapy. Despite the advent of autologous peripheral blood stem cell transplantation, most patients ultimately relapse with the majority of them being resistant to standard chemotherapy. Moreover, although significant proportions of newly diagnosed patients respond to chemotherapy some display primary drug resistance. Acquired drug resistance develops in myeloma as a result of prior chemotherapy. The mechanisms of resistance in myeloma and other haematological malignancies are diverse. For example, overexpression of multidrug transporters and anti-apoptotic proteins and activation of DNA repair pathway are a few that have been described in myeloma.

The treatment of myeloma has been revolutionised over recent decades due to the development of autologous peripheral blood stem cell transplantation, novel therapies and understanding of the basic biology and genetics of myeloma. Conventional cytotoxic chemotherapy has always been the standard therapy for myeloma. Induction chemotherapy with combination of steroid, anthracyclines and vincristine followed by high dose melphalan and rescue with autologous peripheral blood stem cells is the standard therapy for those patients who can tolerate high dose therapy. It results in improved overall survival and a proportion of these patients are cured. The advent of allogeneic stem cell transplantation, proteasome inhibitor and other treatments have not only improved response rates, but also led to the identification of new pathways in the biology of myeloma.

Myeloma is a malignant clonal proliferation of plasma cells constituting more than 10% of bone marrow cells (or biopsy of a tissue with monoclonal plasma cells). It is associated with production of monoclonal immunoglobulins known as paraprotein, M-protein or M-component in the serum or urine. Complications of the disease include hypercalcemia, renal insufficiency, anaemia, immune paresis and lytic bone lesions. It is often preceded by a pre-malignant condition known as MGUS (monoclonal gammopathy of unknown significance), which is an asymptomatic condition associated with monoclonal proliferation of plasma cells of constituting <10% of bone marrow cells. The average incidence of myeloma in U.S. and Australia is 3- 5/100,000, amounting to approximately 1% of all cancers. It is more prevalent in African American men than Caucasian men (8-9 vs. 4.3 per 100 000) and uncommon in China (< 1/100,000) (IARC Worldwide Cancer Incidence Statistics). The male to female ratio is 3:2 and incidence rises with age.

Myeloma is a genetically heterogenous disease consisting of ploidy and structural abnormalities. Approximately 70% of myeloma patients have translocations involving the IgH locus at 14q23, resulting in constitutive expression of cyclin D1 or D3, fibroblast growth factor receptor 3 (FGFR3), myeloma set domain (MMSET), MAF or MAFB. Advanced disease is associated with N-RAS or K-RAS mutations, MYC dysregulations, TP53 mutations, bi-allelic deletion of p18 and/or inactivation of the RB gene. Chromosome 13 deletions and amplification of chromosome 1 are associated with transition from MGUS to myeloma and poor prognosis. Despite the discoveries of these genetic abnormalities in myeloma, the molecular mechanisms that determine individual patient's response to cytotoxic and novel agents are not known.

The overall pattern of disease is characterized by recurring phases of activity, response to therapy, plateau phase, relapse and progressive disease. Prior to the introduction of alkylating agents, the median survival of patients with myeloma was less than a year. With alkylating agents and prednisone as initial therapy, approximately 40%-60% of patients respond and median survival increased to 3 years. Combinations of alkylating agents, vincristine and doxorubicin improved response rates to 60%, compared with 53% for melphalan and prednisone, but had no significant improvement in overall survival. In particular, the combination of vincristine, dexamethasone and doxorubicin (VAD) produced an overall response rate of 84%, with 28% of patients entering complete remission. Maintenance therapy with interferon-α had marginal effect in terms of prolonging response and survival. The observation of the dose effect of melphalan led to the development of a new approach using high dose melphalan and rescue with autologous stem cell transplantation. In comparison to combination therapy, high dose chemotherapy led to better 5-year event free survival and overall survival, of 28% and 52% respectively. The complete response rate was 30-40%. Tandem autologous stem cell transplantation compared to single transplant was associated with modest improvement in 7-year event free survival from 10% to 20%, overall survival from 21% to 42% with a greater effect in those who did not achieve a very good partial response within 3 months of the single transplant. Thus conventional chemotherapy is not curative and even autologous stem cell transplantation is far from being universally effective.

Akylating agents, including melphalan and cyclophosphamide, are the mainstay of therapy for myeloma. Alkylating agents kill tumour cells by damaging DNA, and resistance to these agents is mediated by enhanced repair of DNA inter-strand crosslinks. The Fanconi anaemia/BRCA pathway and overexpression of O6-methylguanine-DNA methyltransferase (MGMT) are also potential mechanisms of resistance to alkylating agents in myeloma.

Recently, thalidomide has been used as part of induction, maintenance and salvage therapy for relapsed myeloma. Thalidomide as post-transplantation maintenance therapy was shown to improve the complete response rate, event free survival and probably overall survival. As a single agent for relapsed disease, the overall response rate to thalidomide is in the order of 32%, with approximately 10% having at least very good partial response or complete response. Combination of dexamethasone and thalidomide as induction therapy in newly diagnosed myeloma yielded an improved overall response rate of about 64%—about as good as VAD.

A significant proportion of refractory and relapsed myelomas respond to proteasome inhibitors (1). Bortezomib is the first proteasome inhibitor to be approved for use in relapsed multiple myeloma based on its remarkable efficacy in this context. In a randomised trial, 669 patients with relapsed myeloma were treated with either Bortezomib or high dose dexamethasone (1). Those patients who were treated with Bortezomib achieved higher response rates, longer time to progression and longer survival. The overall response rate of Bortezomib was 38%, compared with dexamethasone 18%, p<0.001. The complete response rates were 6% and <1% respectively and the one-year survival rates 80% and 66% respectively.

Although the optimal therapy for relapsed disease is not well established, dexamethasone is commonly used as salvage therapy and its activity accounts for most of the antitumour effect of VAD. Nevertheless, it is clear that Bortezomib is effective in refractory cases, as found in a number of phase I and II trials with similar overall response rate of ˜30% (2, 3). Bortezomib in combination with doxorubicin, melphalan, VAD, dexamethasone, thalidomide and cyclophosphamide has also been tested in phase II studies, showing promising results. Despite its efficacy and increasing use, its relevant mechanism of action has not been elucidated. Bortezomib is a relatively well-tolerated drug but even so, a significant proportion of patients do develop side effects, chiefly fatigue, nausea, diarrhoea, thrombocytopenia, anaemia and peripheral neuropathy. Primary resistance to Bortezomib occurs in about 60% of relapsed patients. Acquired resistance occurs with repeated use and again, the mechanism involved is unknown.

P-glycoprotein (P-gp), a product of ABCB1 (or MDR1) gene, is one of the mechanisms of acquired drug resistance in myeloma. It is an ATP-dependent multidrug efflux pump with a spectrum of substrates including anthracyclines, Vinca alkaloids, etoposide (epipodophyllotoxins) and taxanes. Its expression in acute myeloid leukaemia is associated with lower remission rate and poor prognosis. Its expression is low in untreated myeloma and significantly increased after treatment with doxorubicin and vincristine. This phenotype correlates with increasing cumulative dose of its substrate drugs. In vitro studies had shown that P-gp inhibitors such as verapamil and cyclosporin could reverse resistance by directly inhibiting the drug efflux activity of P-gp. However, subsequent clinical trials have shown no clinically significant improvement in response with the addition of P-gp inhibitors and severe toxic effects with increased dose of verapamil or cyclosporin used in this role. Valspodar (PSC833), a cyclosporin D derivative, is 5 times more potent in terms of inhibitory activity on P-gp. In a recent phase III randomised control trial, addition of Valspodar did not improve outcome, and again increased toxicity.

Interaction with extracellular matrix and stromal cells enhances the survival of myeloma cells and may contribute to primary and acquired drug resistance. This stromal interaction is associated with an array of events that promote the survival of myeloma cells. Briefly, the interactions are mediated by the binding of integrin receptors, VLA4 and VLA5 to fibronectin. This leads to induction of p27Kip1 protein, inhibition of cyclin A and E associated kinase activity, induction of c-IAP and Bim and hence, G1 growth arrest. This was referred to as cell adhesion mediated-drug resistance (CAM-DR). Adhesion of myeloma to bone marrow stromal cells activates mitogen activated protein kinase (MAPK) and nuclear factor-κB (NF-κB) which has multiple downstream events, including autocrine and paracrine secretion of cytokines. Resistance to dexamethasone-induced apoptosis in myeloma has been attributed to autocrine and paracrine secretion of insulin growth factor-1 (IGF-1), IL-6 and activation of NF-κB.

Primary and secondary resistance to Bortezomib are observed in the clinical setting. In vitro studies have shown that overexpression of Hsp 27 might contribute to Bortezomib resistance whereas transmembrane drug transporters are unlikely to do so. Currently, there is no predictor of Bortezomib response and cytogenetic abnormalities do not influence the outcome of Bortezomib therapy.

Despite of the advance of prognostic risk stratification for myeloma based on serum β₂ microglobulin, serum albumin, plasma cell labelling index, cytogenetic abnormalities and genetic profiling, a clinically useful marker for specific therapeutic response or drug resistance is still lacking.

### SUMMARY OF THE INVENTION

Broadly stated, the invention stems from recognition that the level of activity of the unfolded protein response in cancer cells may provide an indication of the likely resistance or sensitivity of the cancer cells to proteasome inhibitor treatment. Proteasome inhibitors disrupt the unfolded protein response. Without being limited by theory, it is believed that lower activity of this response reflects lower dependence on the response by the cancer cells rendering the cancers resistant to proteasome inhibitor treatment. Conversely, a higher level of the unfolded protein response activity in cancers which produce and/or secrete greater levels of protein indicates a greater dependence on the response rendering the cancer cells more sensitive to the effects of proteasome inhibitor treatment.

In one aspect of the invention there is provided a method for predicting response to a proteasome inhibitor in the prophylaxis or treatment of a cancer in an individual, comprising:

providing a sample of cancer cells of the cancer from the individual;

evaluating the level of at least one molecule in the cancer cells associated with the unfolded protein response of the cancer cells, to provide test data indicative of the level of activity of the unfolded protein response; and

using the test data to predict the response of the cancer cells to the proteasome inhibitor.

In one or more forms, the method may further comprise comparing the test data to reference data to predict the likely response of the cancer cells to the proteasome inhibitor.

The evaluation of the level of the molecule may comprise assaying for the molecule. The molecule can for example be a protein or a nucleic acid (eg., mRNA or cDNA) encoding the protein or a fragment thereof. The assay used for the determination of the level of the molecule can comprise any suitable assay protocol including enzyme based or nucleic acid amplification protocols. In a particularly preferred embodiment, nucleic acid encoding for the molecule is amplified and the amount of the amplified product obtained is measured.

Hence, a method embodied by the invention may further comprise the steps of:

amplifying cDNA target nucleic acid encoding for the molecule utilising a process involving thermocycling and primers to obtain amplified product; and measuring the amount of the amplified product.

Typically, the molecule will be a component of a signaling pathway of the unfolded protein response selected from the IRE1/XBP-1 and ATF6 signaling pathways. In one form, the molecule is a regulatory factor which mediates activity of the unfolded protein response. Typically, the regulatory factor is a transcription factor.

The predicted response to the proteasome inhibitor facilitates the making of decisions on treatment of the cancer, such as whether treatment with the proteasome inhibitor is likely to be effective or whether a different therapeutic treatment should be administered to the individual.

Accordingly, in another aspect of the present invention there is provided a method for determining a treatment for cancer in an individual, comprising:

providing a sample of cancer cells of the cancer from the individual;

evaluating the level of at least one molecule in the cancer cells associated with the unfolded protein response of the cancer cells, to provide test data indicative of the level of activity of the unfolded protein response; and

using the test data to select a treatment for treating the cancer.

Similarly, the determined level of activity of the unfolded protein response of the cancer cells facilitates determination of a prognosis of the cancer in response to, or absence of, treatment of the cancer cells with a proteasome inhibitor.

The cancer can be any cancer for which the administration of a proteasome inhibitor is a possible option for treatment of the cancer. Likewise, the individual can be a mammal such as a rodent, rabbit, guinea pig, primate or other mammalian species. Typically, the individual will be a human being.

The features and advantages of the present invention will become further apparent from the following detailed description of embodiments thereof together with the accompanying drawings.

### BRIEF DESCRIPTION OF THE ACCOMPANYING DRAWINGS

The file of this patent contains at least one drawing executed in color. Copies of this patent with color drawings will be provided by the Patent and Trademark Office upon request and payment of the necessary fee.

FIG. 1 is a diagram showing the molecular effects of Bortezomib.

FIG. 2 shows a XBP-1 nucleic acid fragment (SEQ ID. No. 1) and local nucleic acid homologies in an intron sequence (SEQ ID. No. 2) for the design of primers specific for the spliced and unspliced forms of the XBP-1 protein.

FIG. 3A is a diagram showing the location of primers for real time PCR for total XBP-1 mRNA.

FIG. 3B shows analysis of PCR amplification products on 10% polyacrylamide gel demonstrating distinct separation of unspliced and spliced XBP-1 amplicons, with no evidence of primer dimer formation.

FIG. 4 shows quantitation of the ratio of spliced:unspliced XBP-1 PCR amplicons generated from mixed unspliced:spliced XBP-1 plasmid template in the ratios shown.

FIG. 5 shows graphs illustrating the effect of Bortezomib on the proliferation of human myeloma cell lines (A) U266; (B) RPMI-8226; (C) KMS-11; (D) H929; and (E) OPM2.

FIG. 6 is a graph showing real time polymerase chain reaction (PCR) analysis of total XBP-1 mRNA of myeloma and non-myeloma tumour cell lines (error bars show standard deviations of 3 repeats).

FIGS. 7A and 7B show the results of the analysis of ratios of unspliced and spliced XBP-1 by polyacrylamide gel electrophoresis, for (FIG. 7A) 6 myeloma cell lines and (FIG. 7B) 13 other tumour cell lines. PCR of each sample was stopped in the late log phase of amplication and analyzed by PAGE. Bands were stained with Sybr I Green and quantified on a Kodak 4000 MM Image station.

FIGS. 8A-8C show graphs illustrating the relationship between Bortezomib resistance and total XBP-1 mRNA levels (FIG. 8A), unspliced XBP-1 mRNA levels (FIG. 8B), and relationship between spliced XBP-1 expression and Bortezomib resistance (FIG. 8C).

FIGS. 9A-9C show graphs illustrating the relationship between XBP-1 mRNA levels and sensitivity to Bortezomib in myeloma cell lines. FIG. 9A: Total XBP-1 mRNA. FIG. 9B: Unspliced XBP-1 mRNA. FIG. 9C: Spliced XBP-1 mRNA. XBP-1 levels were assayed by QPCR. Unspliced:spliced proportions were determined by quantitative PAGE. Bortezomib sensitivity was assayed by proliferation inhibition assay.

FIGS. 10A-C shows graphs illustrating: a comparison of XBP-1 levels and sensitivity to Bortezomib for myeloma cell lines with other cancer types (note log scale on horizontal) (FIG. 10A), and the relationship between Bortezomib sensitivity and total XBP-1 mRNA in non-myeloma lymphoid cell lines (FIG. 10B) and solid cancers (FIG. 10C).

FIG. 11 is a graph illustrating the relationship between total XBP-1 mRNA levels and sensitivity to Bortezomib in protate cancer cell lines (boxed data points) and myeloma cell lines (unboxed data points).

FIG. 12 is a graph showing no relationship between sensitivity to Bortezomib and ratio of unspliced:spliced XBP-1 mRNA in myeloma cell lines.

FIGS. 13A and 13B are diagrams showing the cloning of XBP-1.

FIGS. 14A-14C show graphs illustrating the effects of direct manipulation of XBP-1 levels on sensitivity to Bortezomib. FIG. 14A: Spliced and unspliced XBP-1 levels in RPMI8226 cells transfected to overexpress an unspliced or spliced XBP-1 cDNA. The control is vector only transfectant. FIG. 14B: Sensitivity of the transfectants to Bortezomib. FIG. 14C: Effect of shRNA-mediated knockdown of XBP-1. Controls were shRNA vectors that achieved no reduction in XBP-1 compared to the parent lines.

FIGS. 15A and 15B show graphs illustrating down-regulation of total XBP-1 mRNA levels in Bortezomib-resistant myeloma cell lines, as determined by QPCR (FIG. 15A). Results are shown for resistant sublines during growth with exposure to Bortezomib and 24 and 48 hr after it was washed from the cells. FIG. 15B: Decreased proportion of spliced (active) XBP-1 mRNA in Bortezomib-resistant myeloma cell lines.

FIGS. 16A and 16B show a diagram showing selected components of the Unfolded Protein Response (FIG. 16A) and the down-regulation of components of the UPR in Bortezomib-resistant myeloma cell lines (FIG. 16B). Levels were assayed by immunoblotting with polyclonal or monoclonal antibodies specific for the phosphorylated form of eIF2α, the chaperone BiP, and the transcription factor ATF6, the kinase inhibitor p58^{INK}. GAPDH is a loading control.

FIG. 17A-17D show graphs illustrating the relationship between pre-treatment XBP-1 mRNA levels to response of myeloma patients to Bortezomib: (FIG. 17A) total XBP-1 mRNA, (FIG. 17B) unspliced XBP-1 mRNA, (FIG. 17C) spliced XBP-1 mRNA, and (FIG. 17D) spliced:unspliced XBP-1 mRNA. The latter ratio was determined by quantification of late log-phase XBP-1 PCR products on an Agilent BioAnalyzer and reference to standards consisting of known ratios of PCR products of the spliced and unspliced forms of the mRNA. The patients were grouped according to response as defined by the European Group for Blood and Marrow Transplantation (EBT) criteria as: no response (NR), a minimal response (MR), partial response (PR), or very good partial response (VGPR).

FIG. 18 is a graph illustrating the relationship between total XBP-1 mRNA levels and time to progression for myeloma patients treated with Bortezomib, which is an independent measure of treatment outcome.

### DETAILED DESCRIPTION OF EMBODIMENTS OF THE INVENTION

The ubiquitin-proteasome pathway is the principle pathway for intracellular degradation of proteins. Misfolded or damaged proteins are recognized by the ubiquitin enzymes, which mediate the sequential binding of ubiquitin moieties to the proteins to form a covalently bound polyubiquitin chain. The polyubiquitin chain binds to the 19S proteasome subunits of the 26S proteasome, which recycle the ubiquitin, unfold the proteins and allow their entry into the 20S cylindrical core of the proteasome for proteolysis. The core of the proteasome has three major proteases, “chymotryptic-like”, “tryptic-like” and postglutamyl proteases. Many proteins involved in the control of cell cycle, transcriptional activation, apoptosis and cell signalling are degraded in this way.

The unfolded protein response assists the proper folding of proteins and prevents the accumulation of misfolded proteins in the endoplasmic reticulum. The mammalian unfolded protein response consists of signalling pathways involving three endoplasmic reticulum transmembrane proteins: IRE1 (an endonuclease, Inositol-Requiring Enzyme 1), PERK (protein kinase R-ER related kinase) and ATF6. As unfolded proteins accumulate in the endoplasmic reticulum, the molecular chaperone BiP (also known as GRP78) dissociates from the endoplasmic reticulum membrane-bound enzyme and bind to the hydrophobic surfaces of these proteins.

IRE1 in the absence of BiP auto-transphosphorylates in the cytoplasmic domain and its endoribonuclease becomes activated. In a unique extranuclear splice reaction, it cleaves a 26 base intron from XBP-1 mRNA, producing an open reading frame shift that yields a longer polypeptide (4). The spliced XBP-1 protein is a 54 kD basic leucine zipper transcription factor, which consists of a DNA binding domain in the N-terminus and a transactivation domain in the C-terminus. It induces the promoter elements of multiple stress response genes including, DnaJ/Hsp40-like genes, p58IPK, ERDj4, HEDJ, EDEM, protein disulfide isomerase-P5, ribosome-associated membrane protein 4 (RAMP4) and BiP (5). Unspliced XBP-1 mRNA has a shorter reading frame that is translated to a 30 kDa protein that lacks the transactivation domain in C-terminus. It retains the DNA binding and dimerization domains and has been shown to act as a dominant negative (6). Unspliced XBP-1 has been shown to be unstable and degraded rapidly by the proteasome pathway.

The second transmembrane component of the unfolded protein response, PERK, is a member of the eIF2α family of kinases. Its auto-phosphorylation in response to endoplasmic reticulum stress leads to attenuation of protein translation (7). The third endoplasmic reticulum transmembrane component is ATF6, which is, like XBP-1, a basic leucine zipper transcription factor. It is expressed constitutively as an inactive form. Endoplasmic reticulum stress leads to its activation by the proteolytic cleavage of its cytoplasmic N-terminus. ATF6 induces the transcription of XBP-1 as well as other stress response genes (8). ATF6 alone, however, is not adequate for plasma cell differentiation and immunoglobulin production which also requires the IRE1 induced splicing of XBP-1 mRNA (9).

The unfolded protein response is dependent on the ubiquitin-proteasome pathway for disposal of misfolded protein. Proteasome inhibition disrupts the unfolded protein response by inhibition of proteolysis and accumulation of misfolded proteins in the endoplasmic reticulum. It has been reported that the consequence of exposure to the proteasome inhibitor MG132 is the induction of stress response genes, such as BiP and CHOP (6). Bortezomib treatment leads to the rapid rise and disappearance of the spliced XBP-1 protein and the progressive increase of the unspliced/inactive form of XBP-1. The early rise of spliced XBP-1 protein may be a response to Bortezomib induced stress. However, the reason for the disappearance of the spliced XBP-1 protein is not known.

Blimp-1 and XBP-1 are essential transcription factors in plasma cell development. XBP-1 is downstream to Blimp-1 (10, 11) and drives plasma cell differentiation by repression and activation of multiple genes (11). One of its actions is the repression of PAX5, which is a direct repressor of XBP-1.

Plasma cells and their malignant counterparts are capable of producing high levels of immunoglobulins. Hence, plasma cells also produce high levels of molecular chaperones that ensure proper translation and folding of proteins, and so are highly dependent on the unfolded protein response. XBP-1 is a central regulator of this response and was first identified by its ability to bind to the cyclic AMP response element (CRE) sequence in the gene encoding MHC class II DRα molecule. Although ubiquitously expressed, the level of XBP-1 is highest in plasma cells. Xbp-1 deficient mice are devoid of plasma cells, impaired in immunoglobulin secretion and unresponsive to T independent and T-dependent antigens, despite having normal B cells and germinal centres. XBP-1−/− B cells retain the functions of class switch recombination, cell surface activation marker expression and cytokine secretion, but have decreased expression of J-chain and increased expression of c-myc, indicative of a blockade in plasma cell differentiation. Conversely, ectopic expression of XBP-1 in BCL1-3B3 cells (an activated B cell line that can be driven to early plasma cell stage) leads to further differentiation with decrease in CD44 levels and emergence of Syndecan-1 (CD138) positive cells similar to plasma cells (12).

The splicing of xbp-1 mRNA has previously been reported to be associated with plasma cell differentiation in mice (13). In that study, IL4 and CD 40 stimulation of primary B cells and activated B cells led to the elevation of spliced xbp-1 mRNA as well as plasma cell differentiation and immunoglobulin production. Similarly, IL-2 and IL-5 stimulation of the BCL1 mouse cell line resulted in splicing of xbp-1, induction of unfolded response genes grp78 and grp94, as well as plasma cell differentiation, immunoglobulin production, upregulation of blimp-1 and downregulation of c-myc. It was also observed that spliced but not unspliced xbp-1 protein reconstituted IgG2b expression in xbp1−/− mice. Enforced expression of unspliced and spliced xbp-1 increased IL-6 secretion in activated mouse splenic B cells (both wild type and xbp−/−).

The role of XBP-1 in malignant plasma cells (eg. multiple myeloma) is unclear. However, as in plasma cells, XBP-1 is highly expressed in myeloma. Knockdown of xbp-1 mRNA in mouse myeloma cell J558 by siRNA sensitised the cells to stress-induced apoptosis (6). Nakamura et al (14) have also reported that increased expression of spliced XBP-1 was associated with poor survival in 22 myeloma patients.

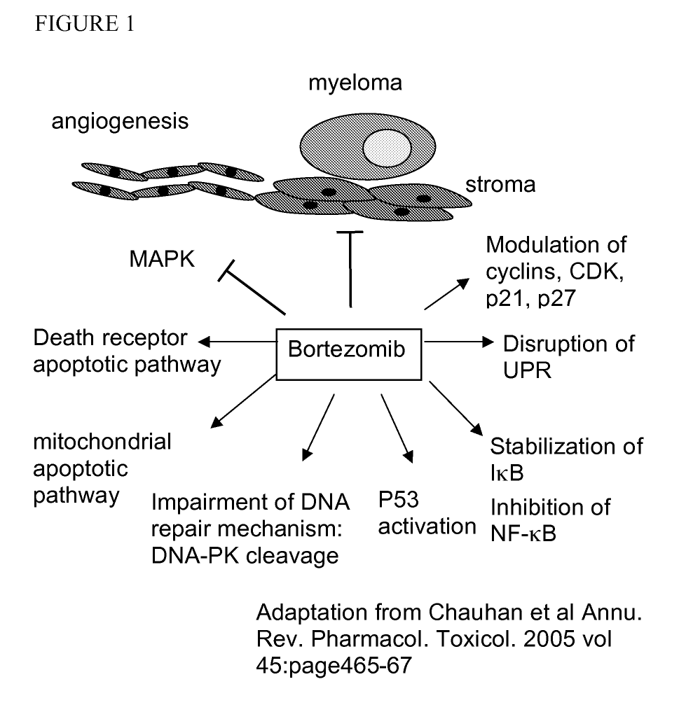

Bortezomib (Velcade) selectively and reversibly inhibits the chymotryptic protease activity of the 26S proteasome, and affects multiple intracellular signalling pathways but the mechanism by which Bortezomib induces apoptosis is unknown. Bortezomib is known to suppress NF-κB activity by inhibiting IκBα degradation. NF-κB activation is important for the survival, cell-cell interaction and drug resistance of myeloma. For example, direct inhibition of NF-κB by PS-1145 is insufficient to completely inhibit the proliferation of myeloma cells (15). As such, NF-κB suppression is not the sole pathway of Bortezomib cytotoxicity and provides no clear rationale for the observed sensitivity of myelomas to the drug. Bortezomib's selective cytotoxicity for myeloma and prostate cancer was first demonstrated in the NCI panel of human tumours. The reason behind this sensitivity is unknown. However, both cell types are highly secretory. That Bortezomib worked was counterintuitive at first sight. However, its efficacy and tolerability led to rapid clinical development and therapeutic approval in the United States for relapsed myeloma. It is of interest that glandular/secretory cancers, specifically prostate, pancreatic and breast cancers have sometimes also responded to Bortezomib in clinical trials. Other molecular events that are affected by proteasome inhibitors are illustrated in FIG. 1.

In accordance with the invention, the increased activity of the unfolded protein response in at least some cancers offers a means for predicting the response of cancer cells to therapeutic treatment with proteasome inhibitors such as Bortezomib.

The cancer may for instance be selected from the group consisting of blood cell cancers, plasma cell malignancies including myeloma cancers, multiple myeloma and plasma cell leukemia, lymphoma, Waldenstrom macroglobulinemia, prostate cancer, breast cancer or any other cancer derived from a cell type whose primary function is secretion, such as glandular cancers.

Besides Bortezomib, other proteasome inhibitors in respect of which a method embodied by the invention may have application in determining resistance or sensitivity of cancer cells to treatment therewith, include leupeptin, calpain inhibitor I, calpain inhibitor II, MG115, MG132, PSI, peptide glyoxal, peptide aldehyde, peptide benzamides, peptide α-ketoamides, peptide vinyl sulfones, peptide boronic acids, NLVS, PS-341, lactacystin, clasto-lactacystin β-lactone, PS-519, epoxomicin, eponemycin, TMC-86A, TMC-86B, TMC-89, TMC-96, YU 101, gliotoxin, HNE(4-hydroxy-2-nonenal), YU 102, NPI-0052, PR-171 and other natural and synthetic proteasome inhibitors. Typically the proteasome inhibitor will be selected from Bortezomib and MG132.

The level of any molecule involved in the unfolded protein response of the cancer cells which is indicative of the level of activity of the unfolded protein response can be evaluated. The presence of higher levels of the molecule will generally be indicative of increased activity or potential activity of the unfolded protein response and in at least some embodiments, will directly correlate with the level of activity of the response. Likewise, lower levels or absence of the molecule will generally be indicative of lower activity of the unfolded protein response. However, in other embodiments the reverse situation may apply. That is, lower levels of the molecule may be indicative of elevated activity of the unfolded protein response while higher levels of the protein are indicative of lower activity of the response. Thus, the evaluated level of the molecule may be used to provide a direct indication of the status of the response.

In this instance, the reference data may consist of a discrete reference value wherein a higher or lower level of the molecule is indicative of sensitivity/likelihood of sensitivity or resistance/likelihood of resistance to the proteasome inhibitor depending on the molecule. Alternatively, the reference data may comprise a range of values which for instance, correlate with increasing likelihood of resistance or increasing likelihood of sensitivity. In another form, the ratio of the level of an inactive form of the molecule relative to the level of an active form of the molecule can be utilised. In this case, the reference data may comprise reference ratios that respectively correlate with decreasing or increasing likelihood of resistance of susceptibility to the proteasome inhibitor.

As will be understood, the reference data may be compiled by measuring the level of the molecule(s) in a range of samples of cancer cells from a group of individuals suffering from the relevant cancer and associating level of sensitivity or resistance of the cancer cells to treatment with the proteasome inhibitor with increasing levels of the molecule(s) in the various samples of cancer cells.

The molecule may for instance be a stress response gene product or regulatory factor such as a transcription factor (eg. XBP-1, Blimp-1), protein disulphide isomerase-P5 or other molecule involved in the unfolded protein response as described above such as RAMP4 and BiP. Similarly, levels of degradation products of the foregoing proteins or mRNA encoding for proteins or polypeptides involved in the unfolded protein response can be measured to provide an indication of the level of the expressed protein or polypeptide. In one or more embodiments, the molecule(s) may be selected from XPB-1, ATF-6, BLIMP-1, DnaJ/Hsp40-like proteins, p58IPK, ERDj4, HEDJ, EDEM, protein disulfide isomerase-P5, ribosome-associated membrane protein 4 (RAMP4) and BiP. The molecule will generally be XBP-1.

The total abundance of the expressed protein or mRNA encoding active and inactive forms of the protein can be determined. Surprisingly, it has been found that the abundance of unspliced XBP-1 mRNA correlates with the level of activity of the unfolded protein response in myeloma cancer cells. Rather, it would have been expected that determination of the level of spliced mRNA (coding for the active form of XBP-1) would reflect activity of the unfolded protein response and thereby be predictive of the response of the cancer cells to proteasome inhibitor treatment. As a consequence, it not necessary to distinguish the two forms of the protein mRNA as the unspliced form of the mRNA is predominant.

Hence, in one or more embodiments, total levels of XBP-1 protein or mRNA encoding for the protein may be evaluated. In another embodiment, the level of spliced or unspliced XBP-1 mRNA may be evaluated.

A prediction of the likelihood of the response to the cancer cells by the proteasome inhibitor can allow assessment of whether to treat the individual with the proteasome inhibitor, or for instance, whether to increase or decrease a dosage level of the inhibitor depending on the predicted susceptibility of the cancer cells to the inhibitor. If the prediction is that the cancer cells are likely to be resistant to treatment with the inhibitor, an alternative treatment may then be tailored or selected for the individual. For instance, other cancer treatments conventionally utilised may then be considered by the attending physician such as combination therapy with alkylating agents, vincristine and doxorubicin, or melphalan and prednisone.

As will also be understood, an adverse prediction indicative of the likelihood of a poor response to a proteasome inhibitor may indicate a poor prognosis for the cancer. Alternatively, a prediction that the cancer cells are likely to be sensitive to the proteasome inhibitor may indicate a positive prognosis.

Enzyme based assays suitable for detection of proteins and polypeptides in accordance with the invention include radioimmunoassay (RIA) and ELISA assays (eg., see Handbook of Experimental Immunology, Weir et al., Vol. 1-4, Blackwell Scientific Publications 4^{th} Edition, 1986, and any subsequent updates thereof). Such assays include those in which a target molecule is detected by direct binding with a labelled antibody, and those in which the target antigen is bound by a first antibody, typically immobilised on a solid substrate (eg., a microtitre tissue culture plate fabricated from a suitable plastics material such as polystyrene or the like), and a labelled second antibody specific for the first antibody for forming a target molecule-first antibody-second antibody complex that is detected by a signal emitted by the label. Sandwich techniques in which the antigen is immobilised by an antibody for presentation to a labelled second antibody specific for the molecule are well known. An antibody can be bound to a solid substrate covalently utilising commonly employed amide or ester linkers, or by adsorption. Optimal concentrations of antibodies, temperatures, incubation times and other assay conditions can be determined with reference to conventional assay methodology and routine experimentation.

Antibodies specific for the target molecule can be polyclonal or monoclonal. Preferably, the antibody will be monoclonal antibody. The production of polyclonal and monoclonal antibodies is well established in the art (eg., see Antibodies, A Laboratory Manual, Harlow & Lane Eds. Cold Spring Harbour Press, 1988, and any subsequent updates thereof). For polyclonal antibodies, a mammal such as a sheep or rat is immunised with the protein, polypeptide or antigenic fragment thereof of interest, and anti-sera is isolated from the mammal prior to purification of antibodies generated against the target molecule by standard affinity chromatography techniques such as Sepharose-Protein A chromatography. Desirably, the mammal is periodically challenged with the relevant antigen to establish and/or maintain high antibody titer. To produce monoclonal antibodies, B lymphocytes can be isolated from the immunised mammal and fused with immortalising cells (eg., myeloma cells) using somatic cell fusion techniques (eg., employing polyethylene glycol) to produce hybridoma cells. Selection of hybrid cells may be achieved by culturing the cells in hypoxanthine-aminopterin-thymidine (HAT) medium, and selected hybridoma cells can then be screened for production of antibodies specific for the target molecule by ELISA or other immunoassay. However, it will be understood that any suitable commercially available antibody can be utilised.

Rather than a complete antibody, binding fragment(s) of antibodies may also be used. The term “binding fragment” of an antibody expressly includes within its scope Fab and (Fab′)₂ fragments obtainable by papain or pepsin proteolytic cleavage respectively, and variable domains of antibodies (eg., Fv fragments) for example linked to suitable peptide support sequences.

An antibody can be labelled with any moiety which by its nature is capable of providing or facilitating production of an analytically identifiable signal allowing the detection of the antibody or complex. For instance, an antibody can be labelled with a radio isotope such as ³²P, ¹²⁵I or ¹³¹I, an enzyme, a fluorescent label, chemiluminescent molecule or for instance an affinity label such as biotin, avidin, streptavidin and the like. An enzyme can be conjugated with an antibody by means of coupling agents such as gluteraldehyde, carbodiimides, or for instance periodate although a wide variety of conjugation techniques exist. Commonly used enzymes include horseradish peroxidise, glucose oxidase, β-galactosidase and alkaline phosphatase amongst others. Detection utilising enzymes is achieved with the use of a suitable substrate for the selected enzyme. The substrate is generally chosen for the production upon hydrolysis of a detectable colour change. However, fluorogenic substrates may also be used which yield a fluorescent product rather than a chromogen. Fluorescent labels include, for instance, fluorescein, phycoerythrin (PE) and rhodamine which emit light at a characteristic wavelength in the colour range following illumination with light at a different wavelength, and any suitable such fluorescent label may be used.

Reverse transcriptase polymerase chain reaction (RT-PCR) is a widely used amplification method enabling detection of RNA coding for proteins or polypeptides of interest and is the preferred means of detection in methods embodied by the present invention. However, any suitable PCR protocol useful in methods described herein can be employed as can any other appropriate nucleic acid amplification protocols.

The invention is described further below by reference to a number of non-limiting examples.

### EXAMPLE 1

### XBP-1 PCR Assay

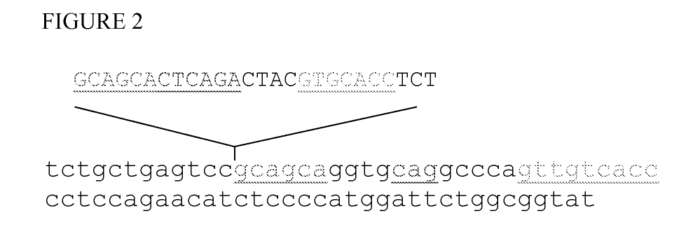

XBP-1 mRNA consists of 5 exons and 1836 base pairs (GenBank NM_{—}005080). Regulated post-transcriptional splicing removes another 26 bp intron at position 541 and produces a shift in the open reading frame, which is longer in the spliced form. The 26 bp intron (SEQ ID. No. 2) is highly homologous to the adjacent sequence downstream (FIG. 2). Primers spanning the 26 bp intron amplify both forms with similar efficiency and the products can be readily distinguished by polyacrylamide gels electrophoresis.

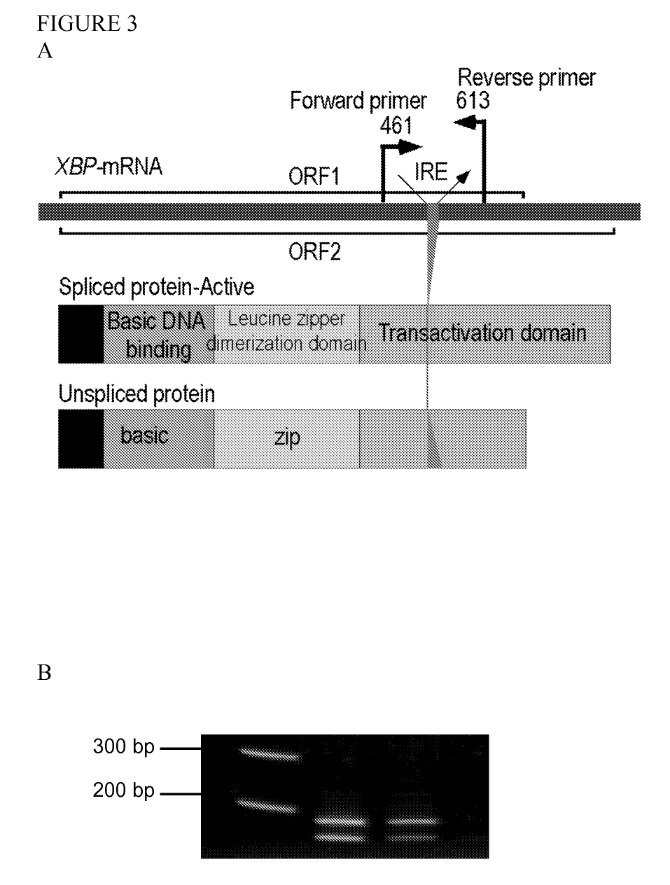

The polymerase chain reaction (PCR) assay for XBP-1 described below is a two-step process. Firstly, total XBP-1 mRNA is determined by quantitative real time PCR (FIG. 3A). The second step involves comparing the abundance of the spliced and unspliced XBP-1 forms at late log phase of PCR by gel electrophoresis and densitometry on a CCD camera system (FIG. 3B).

1.1 Quantitation of Total XBP-1 mRNA by Real Time PCR

The quantitation of total XBP-1 mRNA was performed by reverse transcriptase-quantitative real time RT-PCR assay. Briefly, the primers used were: forward 5′-ggagttaagacagcgcttgg-3′ (SEQ ID No. 3) and reverse 5′-gtcaataccgccagaatcc-3′ (SEQ ID No. 4) at sites 461 and 613, based on sequence of GeneBank NM_{—}005080.

Total RNA was extracted from cells using Tri Reagent (isophasic guanidine isothiocyanate:phenol, MRC) according to the manufacturer's protocol (TRI Reagent—RNA, DNA, protein isolation reagent. Manufacturer's protocol (1995), Molecular Research Center, Inc. Cincinnati, Ohio). RNA was treated with DNase I (Ambion) for removal of genomic DNA contamination. The quality and quantity of RNA was checked by gel electrophoresis and spectrophotometry at 260 nm. A 2.5 μg aliquot of RNA was used for first strand cDNA synthesis with SuperScript™ III Reverse Transcriptase (Invitrogen). Real time PCR was performed on the cDNA using a Stratagene Mx3000P, with the following cycling conditions: initial denaturation and activation of the enzyme at 94° C. for 8 min, followed by 35 cycles of 94° C. for 30 s, 64° C. for 30 s, 72° C. for 30 s, 85° C. for 20 s. The PCR reaction was 50 μL in volume consisting of 1.25 units of AmpliTaq Gold DNA Polymerase, 0.2 mM of dNTPs, 50 mM KCl, 10 mM Tris-HCl (pH 8.3), 2.5 mM MgCl₂, 140 nm of each primer, 3% DMSO and SYBR I Green (1:25 000) (Invitrogen). The result was expressed as a ratio of total XBP-1 mRNA normalized to β-Actin. β-Actin was the least variable in human myeloma cell lines compared with other housekeeping genes (BCR, GAPDH, RPL5a, α-tubulin). The quantities of both genes were derived from standard curves. Plasmids containing XBP-1 and β-Actin cDNA were linearized and quantified by spectrophotometry. Ten-fold dilution series were made in the range of 10⁶ to 10³ copies in diluent containing 50 μg/ml tRNA carrier. The primers for β-Actin were: forward 5′-accaactgggacgacatggagaaaa-3′ (SEQ ID No. 5) and reverse 5′-cgcacgatttcccgctcggc-3′ (SEQ ID No. 6).

Melting curves and polyacrylamide gel electrophoresis indicated that the XBP-1 and control assays yielded single products. XBP-1 and β-actin plasmid standards were run with each assay in order to control for any inter-experimental variations in amplification efficiency. The amplification efficiencies of the total XBP-1 and β-actin real time PCR assays were 100% and 90% respectively. The assay was validated by Northern analysis and shown to be repeatable. No products from cDNA synthesis controls in which reverse transcriptase had been omitted were found confirming that the DNase treatment was effective.

1.2 Quantitation of the Ratio of Spliced:Unspliced XBP-1

The proportions of unspliced and spliced XBP-1 were measured either by resolving the PCR products on gel electrophoresis and densitometry using a Kodak CCD camera (Kodak 4000 MM Image Station) or by microelectrophoresis on an Agilent 2100 BioAnalyzer. (Agilent Technologies; www dot agilent dot corn ).

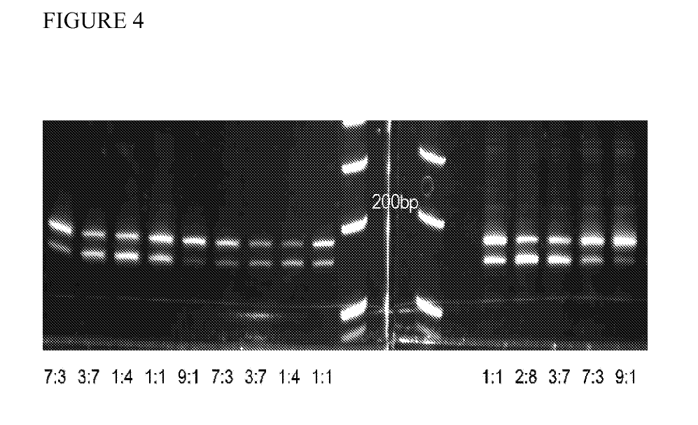

Specifically, PCR products were generated from mixed unspliced:spliced XBP-1 plasmid template in the ratios shown in FIG. 4. PCR was performed using the same conditions as in the total XBP-1 assay described in Example 1.1. Reactions were stopped during late log phase at a point that gave an arbitrarily set threshold fluorescence of 30,000, in order to adjust for the variable starting quantities of templates. When using conventional electrophoretic techniques, the PCR products were resolved on precast 10% TBE polyacrylamide gels (Invitrogen) by electrophoresis at 200 volts for 1 hour. Gels were stained with Sybr I Green 1:10,000 (Invitrogen) for 30 min and its fluorescence imaged in cooled CCD system (Kodak Image Station 4000 mm) for 60 s, using 485 nm excitation and 535 nm emission filters. The unspliced and spliced forms appeared as distinct bands of 192 base pairs and 166 base pairs respectively. The relative net intensity of the unspliced and spliced forms was measured by Kodak software and a molar correction factor of 1.16 (=the size ratio of the PCR products of the two forms) was applied to the spliced form due to its smaller size.

SYBR I Green stain concentration and staining time were titrated on PCR products from standards; 1:10,000 for 30 min proved adequate. Quantitation of PCR products analysed on polyacrylamide gels using Sybr I Green staining and the Kodak 4000 MM Image Station was tested with 10 fold dilutions of the unspliced XBP-1 PCR product from 1.2 ng to 120 ng. All could be detected clearly. The net intensity of fluorescence correlated well with the quantity of DNA.

### EXAMPLE 2

### Relationship Between Bortezomib Sensitivity and XBP-1

2.1 Bortezomib Resistance Assay

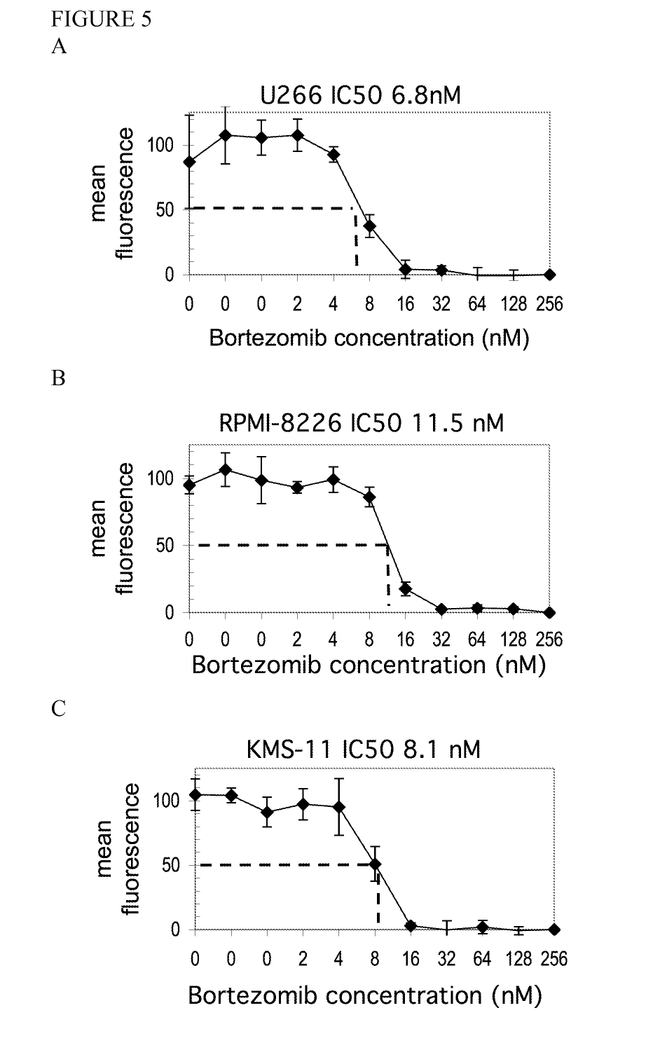

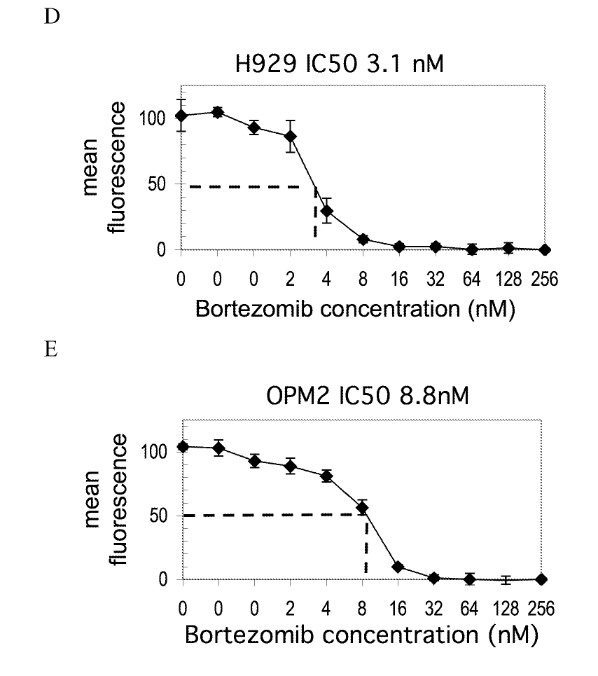

Cytotoxicity assays were performed essentially as described previously (16). For myeloma cell lines, some modifications were made due to their slow growth rate, non-adherence to the culture plate and sensitivity to sparse plating. Briefly, cells were seeded at 10,000 per well in 96-well plates in RPMI-1640 without phenol red (which interferes with detection of fluorescence). Bortezomib (Millennium Pharmaceuticals; Johnson & Johnson Pharmaceuticals) was applied in a concentration series along the long plate axis. After 5 days of proliferation, cells were permeabilised in situ by adding 5% by volume of 21 X Triton X-100 buffer (10 mM TrisHCl pH 7.4, 5 mM EDTA, 0.1% Triton X-100) and Sybr I Green (Invitrogen) 1:4000. The lysate was mixed thoroughly with a multi-channel pipette. One cycle of freeze and thaw was performed to ensure complete lysis. The fluorescence, measured on a plate reader (Wallac), was proportional to the number of viable cells at the end point. The IC50 was determined as the concentration of drug that inhibited proliferation to 50% of the untreated controls (see FIG. 5A-5E).

2.2 Correlation of Bortezomib Resistance With XBP-1 Expression in Human Myeloma Cell Lines

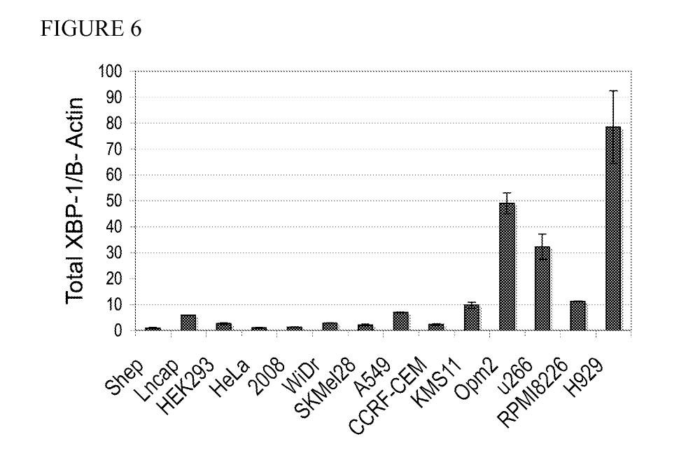

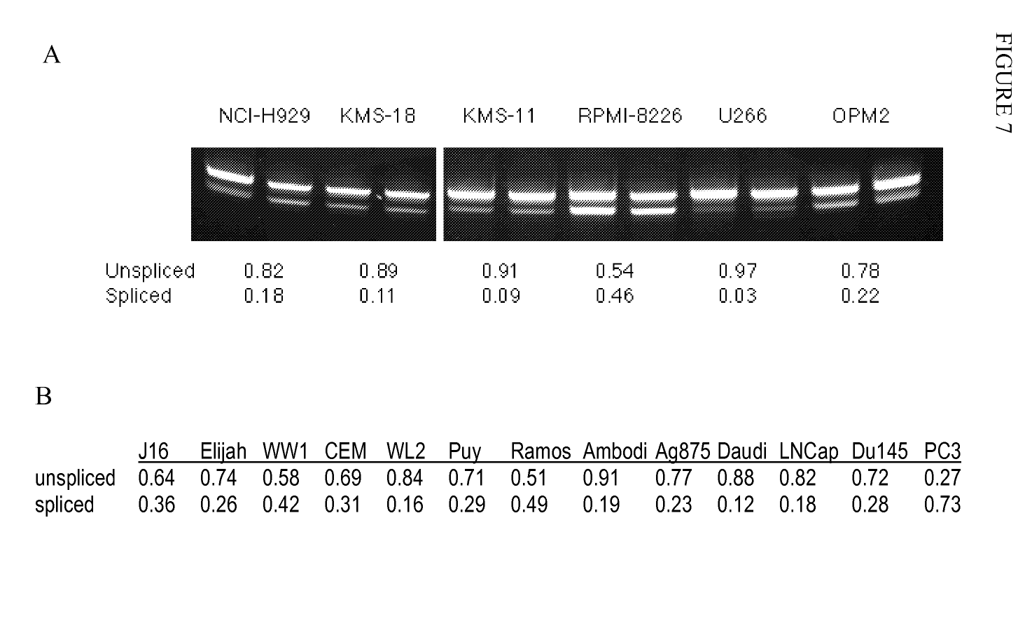

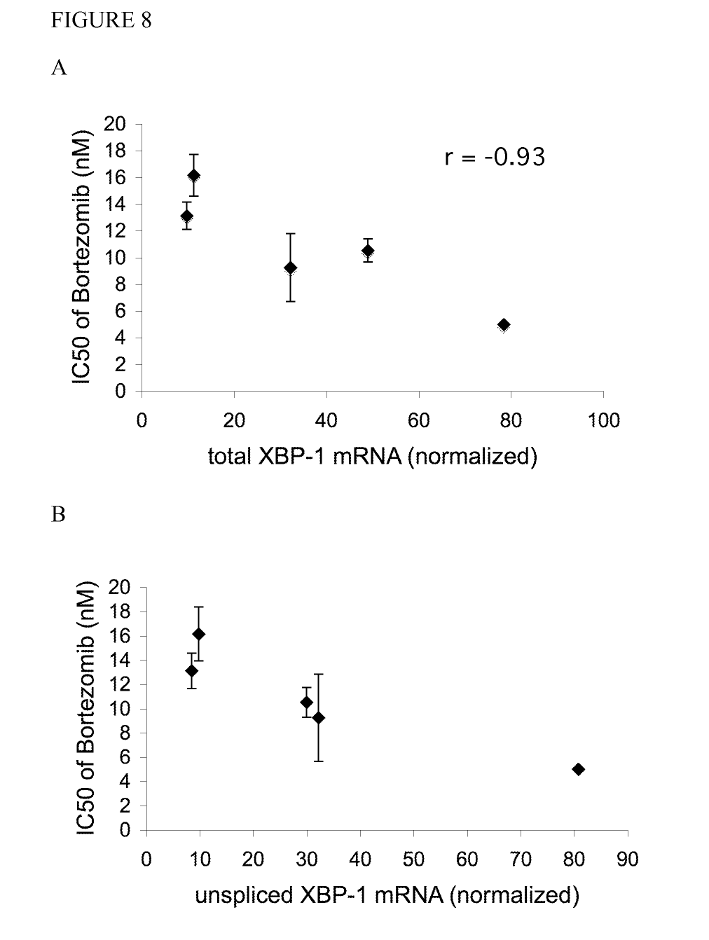

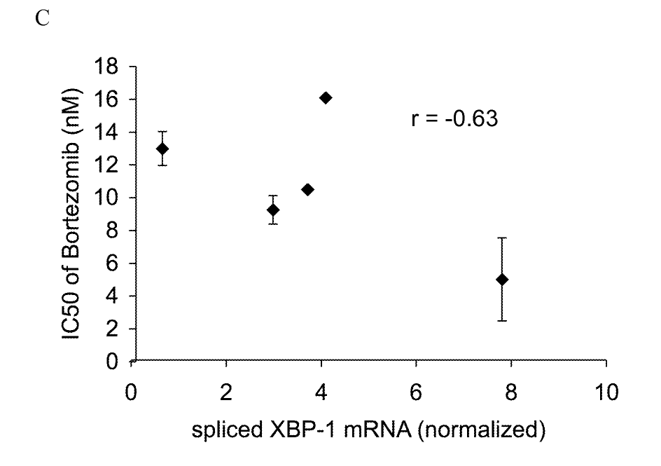

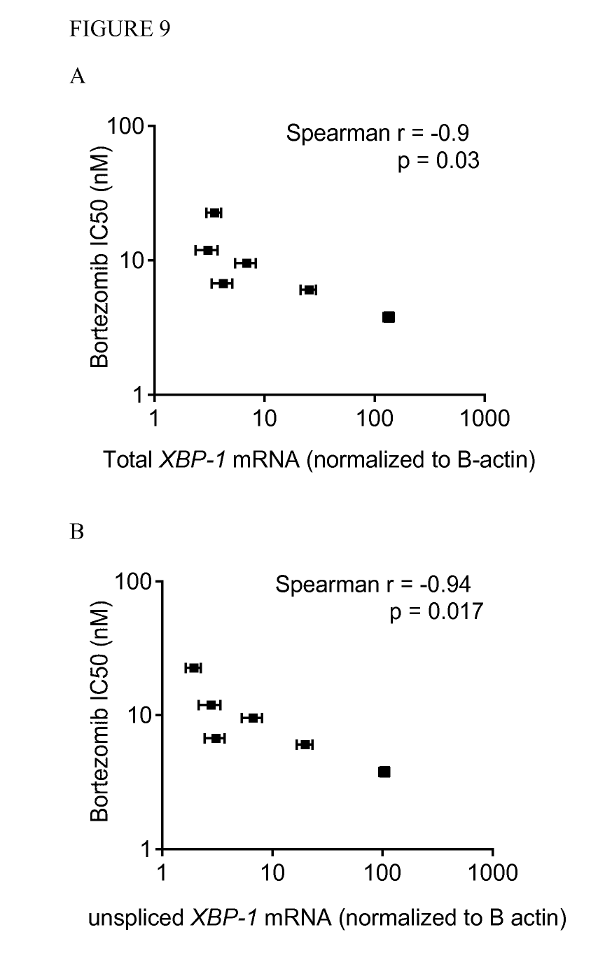

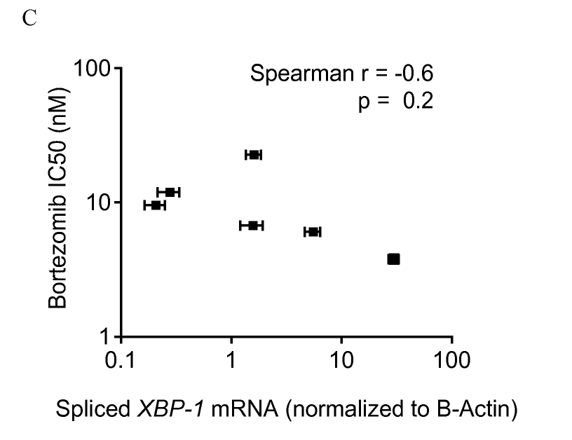

Expression of XBP-1 was variable across myeloma cell lines and other types of cancer cell lines (FIG. 6). The ratio of unspliced:spliced XBP-1 was assessed in myeloma cell lines NCI-H929, KMS-18, KMS-11, RPMI-8226, U226 and OPM2 and unspliced was the predominant form (FIG. 7A). This was also observed to be the case in most of the non-myeloma cell lines tested (FIG. 7B). The total XBP-1 mRNA levels were found to correlate inversely with the resistance of Bortezomib (FIG. 8A-8B) with very similar results for the unspliced form because it accounts for most of the total. Pearson's coefficient was approx. −0.9. The correlation between spliced XBP-1 levels and Bortezomib sensitivity was not as strong (FIG. 8C). A repeat of these experiments provided confirmation of the inverse relationship between the level of total XBP-1 and resistance to Bortezomib (FIG. 9A-9C) Although unspliced XBP-1 protein is the inactive form, its mRNA level reflects the capacity of the unfolded protein response to generate the spliced form when the need arises.

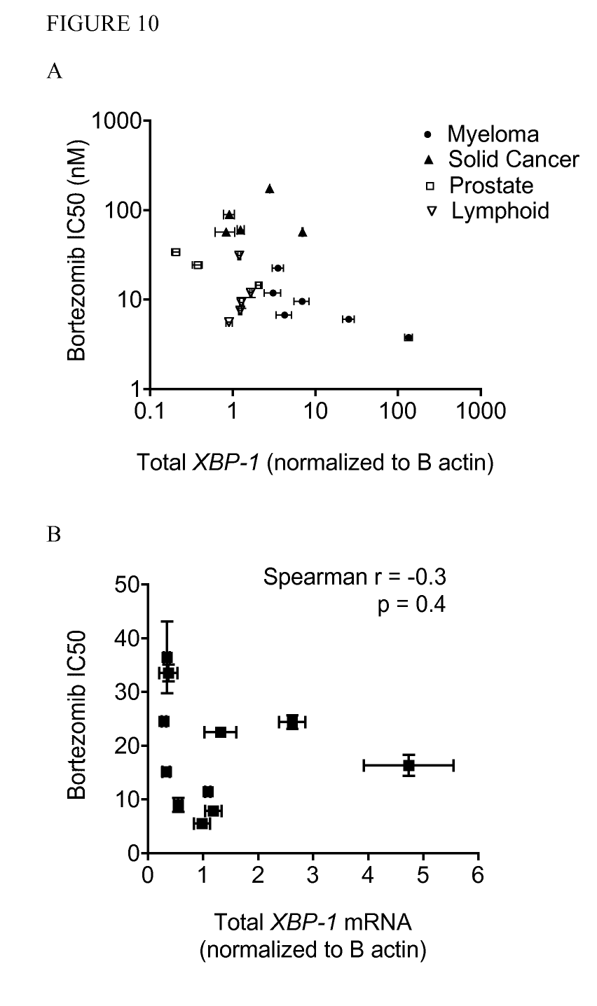

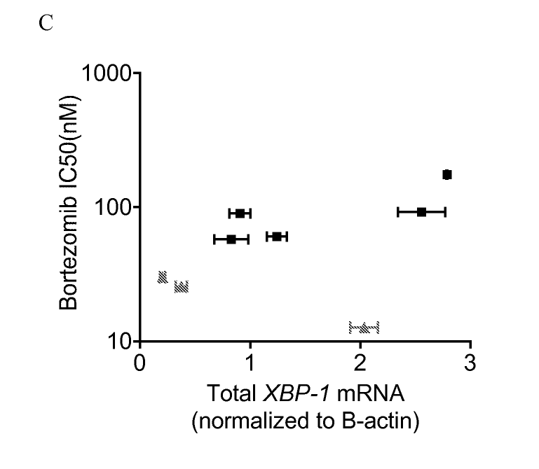

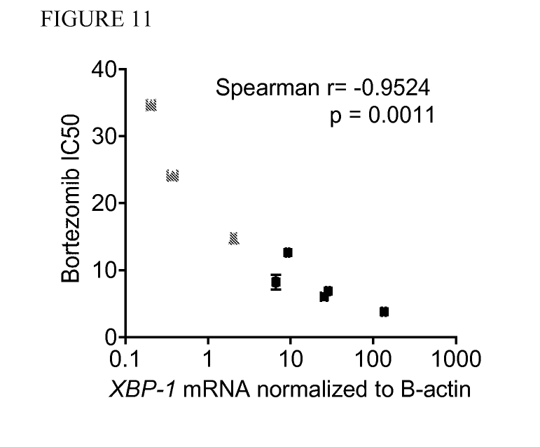

The relationship between XBP-1 levels and sensitivity to Bortezomib is weak for other tumour cell lines, which have lower levels of XBP-1 (FIG. 10A). This is true of both non-myeloma lymphoid lines (FIG. 10B) and solid tumour lines (FIG. 10C) considered as groups. The prostate cancer cell line LNCaP was the most sensitive to Bortezomib of the non-myeloma cell lines and its XBP-1 level was relatively high (FIG. 11). The therapeutic use of Bortezomib is currently being evaluated in patients with prostate cancer.

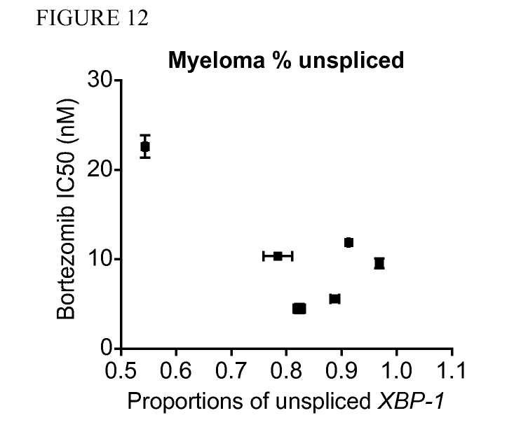

Whilst total XBP-1 mRNA levels were found to correlate inversely with the resistance of Bortezomib, the ratio of spliced to unspliced XBP-1 mRNA in myeloma cell lines is not a useful predictor of sensitivity to Bortezomib in vitro (FIG. 12).

### EXAMPLE 3

### Manipulation of XBP-1 Levels in Vitro for Evaluation of XPB-1 Resistance

Methods for the manipulation of XBP-1 levels in myeloma cells by over-expression of unspliced and spliced XBP-1 and shRNA (short hairpin RNA)—mediated knockdown of XBP-1 in myeloma cell lines are described below for analysis of resistance to proteasome inhibitor treatment. Bortezomib-resistant myeloma cell lines can also be derived (Example 4) to assess for changes in XBP-1 expression as a resistance mechanism.

3.1 Overexpression of Unspliced and Spliced XBP-1 in Cell Lines

3.1.1 Cloning of XBP-1

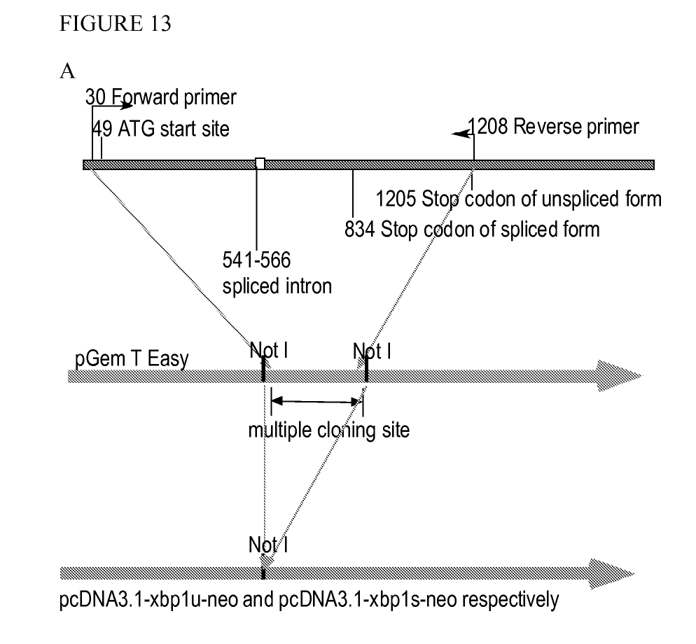

XBP-1 cDNA was obtained by PCR on human myeloma cell line KMS-11 using a high-fidelity polymerase (Pfx Platinum, Invitrogen) and the following primers: sense 5′-cggtgcctagtctggagctatg-3′ (SEQ ID No. 7) and anti-sense 5′-ccatcgatccttagacactaatcagctgggg-3′ (SEQ ID No. 8) based on the sequence of GenBank/NM005080. The amplification product spanned the coding sequence of unspliced and spliced XBP-1 and 19 base pairs upstream from the start codon. PCR product was cloned into pGem T Easy vector. Colonies were screened for the presence of unspliced and spliced XBP-1 by PCR. The sequence was verified by sequencing (Supamac). The XBP-1 cDNA was subcloned into pcDNA 3.1(+) mammalian expression vector (see FIG. 13A).

Sequencing revealed a single nucleotide polymorphism at base 67 which resulted in an amino acid change from alanine to threonine. The SNP was also found (by sequencing PCR products) in another 1 of 5 myeloma cell lines. To correct the SNP at base pair number 67 and to obtain the complete 5′UTR, PCR was performed on human myeloma cell line U266 using a sense primer that starts at the position 1 of the cDNA sequence (5′-ggcgctgggcggct-3′) (SEQ ID No. 9) and anti-sense primer at position 789 (5′-acagagaaagggaggctggt-3′) (SEQ ID No. 10). PCR product was cloned into pGem T Easy vector. The sequence was verified by sequencing. The 5′UTR fragment and the first 253 bp of the coding region was excised with restriction enzymes SphI and Ava I and replaced the corresponding fragment in pGem T Easy-xbpu and pGem T Easy-xbps. The entire XBP-1 fragment was then excised with Not I and inserted into pcDNA3.1(+) mammalian expression vector (Invitrogen) (see FIG. 13B).

3.1.2 Initial Transfection Studies

HEK293 (Human Embryo Kidney epithelial) cells were transiently transfected with the unspliced or spliced XBP-1 cDNA in pcDNA3.1(+) using Lipofectamine (Invitrogen). The transfection efficiency was approximately 48% based on a pcDNA 3.1/GFP control transfection performed in parallel. Northern analysis of the transfectant showed that the constructs were functional.

3.1.3 Transfection of Myeloma Cell Lines

Several lipid reagents were evaluated for transfection of myeloma cell lines, of which Lipofectamine proved most effective. The transfection medium and ratio of lipid:DNA were optimised. Transfection efficiencies for the myeloma lines were tested with pcDNA3.1-GFP plasmid (N. West, Centenary Institute) and analysed by flow cytometry. The transfection efficiencies proved to be variable, ranging from 7.4% for KMS-11 myeloma cells to 5% for 8226 cells. In light of the low transfection efficiencies observed, the cDNAs were transferred to the retroviral vector LZRS-IRES-GFP (17). Amphotropic retroviruses were generated by lipofectamine-mediated transfection of Phoenix-A cells (18) with the retroviral XBP-1 expression constructs.

Briefly, myeloma cell lines, prostate cancer cell lines and lymphoid cell lines were cultured in RPMI-1640 supplemented with 10% foetal calf serum, 100 units/mL penicillin G and 100 μg/mL streptomycin. The other solid cancer cell lines were cultured in DMEM with 10% supplemented calf serum (Cosmic Calf Serum, Hyclone), pencillin and streptomycin. Cells were incubated at 37° in a humidified 5% CO₂ atmosphere. RPMI8226 cells were cultured in RPMI-1640 medium containing 10% (v/v) foetal calf serm, 100 units/mL penicillin G and 100 μg/mL streptomycin, in a 37° C incubator with humidified 5% CO₂ atmosphere. Cells in 24-well plates (10⁵/well) were transduced by adding amphotropic retrovirus supernatant 1:2 to the culture medium, plus 8 μg/mL polybrene, for 8 hr, after which the cells were diluted several fold in fresh medium. Once expanded, transduced cells were isolated by flow cytometry using the GFP marker. Quantitation of spliced and unspliced XBP-1 and determination of sensitivity to Bortezomib was carried out as previously described (Examples 1.1, 1.2 and 2.1, respectively).

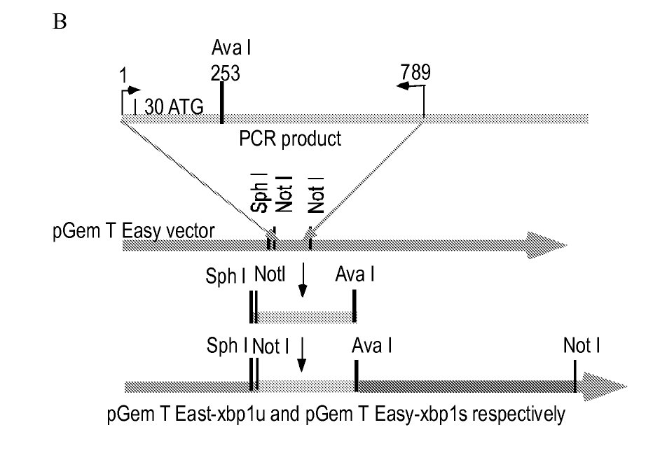

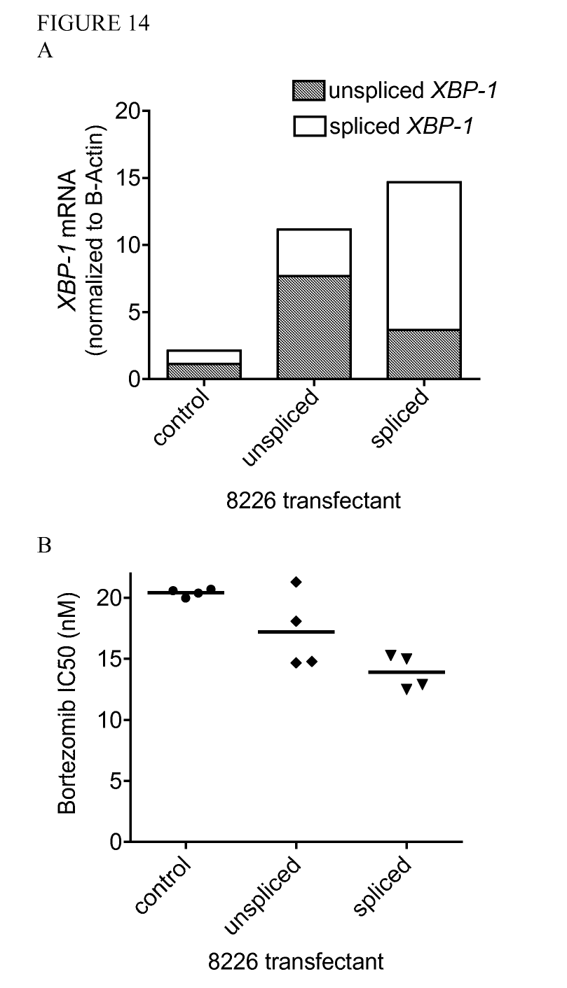

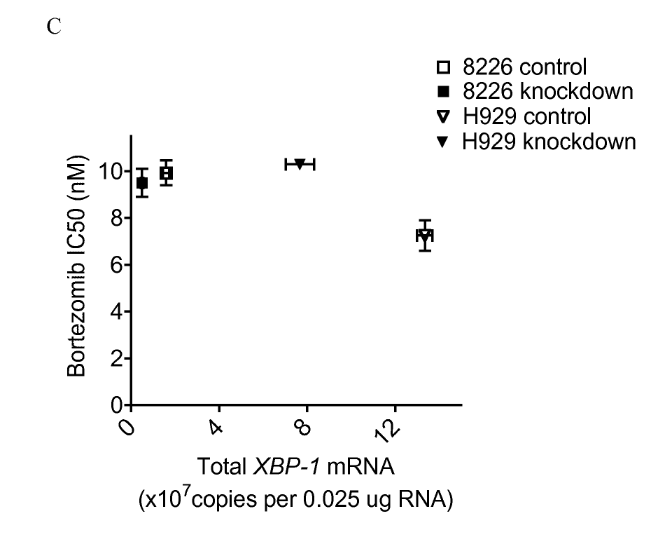

FIG. 14A shows overexpression of unspliced XBP-1 cDNA in RPMI8226 cells transduced with retrovirus expressing unspliced XBP-1 cDNA and overexpression of spliced XBP-1 cDNA in RPMI8226 cells transduced with retrovirus expressing spliced XBP-1 cDNA, as expected. Notably, cells expressing the unspliced XBP-1 cDNA also displayed a higher absolute level of spliced XBP-1 mRNA, presumably because there is a larger pool of unspliced XBP-1 mRNA substrate for IRE1 to splice. Moreover, RPMI8226 cells expressing the spliced XBP-1 cDNA also displayed a considerable increase in the level of unspliced XBP-1. This result likely reflects regulation of the XBP-1 promoter by feedback from its own active, spliced form, a transcription factor, and/or by other components of the UPR.

As shown in FIG. 14B, direct manipulation of XBP-1 levels through overexpression of spliced or unspliced XBP-1 cDNA had had only a small effect on sensitivity of the cells to Bortezomib. Therefore, XBP-1 is a surrogate marker of dependence on the UPR rather than a direct target of Bortezomib.

3.2 Knockdown of XBP-1 Using shRNA (Short Hairpin RNA)

The knockdown of XBP-1 offers a complementary alternative to the over expression studies described in Example 3.1. To perform the knockdown of XBP-1, 5 candidate retroviral shRNAs (short hairpin RNAs) for knockdown of XBP-1 have been obtained in lentiviral vectors as VSVG-pseudotype virus from the MISSION library (Sigma). The vectors carry a puromycin resistance marker meaning that non-XBP-1 control vectors will need to be used in parallel to discern any non-specific effects arising from the drug selection of transduced cells. This is in preference to selection with G418 which requires long periods of selection which could in turn affect the drug resistance properties of the transduced cells. Puromycin selection is rapid, however, compared to G418. Moreover, the principal change in drug resistance properties following puromycin selection is upregulation of P-glycoprotein of which puromycin is a substrate. However, Bortezomib is not a significant P-glycoprotein substrate so there is unlikely to be any confounding influence from that source. The target cell lines H929 which expresses the most XBP-1 (and is most sensitive to Bortezomib) and a low expressing line (e.g. 8226 cells) are used for comparison.

H929 and RPMI8226 myeloma cells were transduced with virus as follows. H929 and RPMI8226 cells were cultured in RPMI-1640 medium containing 10% (v/v) foetal calf serm, 100 units/mL penicillin G and 100 μg/mL streptomycin in a 37° C incubator with humidified 5% CO₂ atmosphere. Cells in 24-well plates (10⁵/well) were transduced by adding lentivirus supernatant 1:10 to the culture medium, plus 8 μg/mL polybrene for 8 hr. Thereafter, medium was replenished with a 50:50 mixture of fresh medium and conditioned medium from the parent cell line. Transduced cells were selected with 2 μg/mL puromycin for 3 days. Surviving cells were expanded for analysis at low passage number. Total XBP-1 and sensitivity to Bortezomib was determined as previously described (Example 1.1 and Example 2.1, respectively).

It was possible to obtain a significant knockdown of XBP-1 in the two myeloma cell lines even though XBP-1 is known to be essential for myeloma cell survival. The knockdown in the H929 cells did result in a decrease in sensitivity to Bortezomib but the change was modest. No such effect on sensitivity to Bortezomib was seen in the RPMI8226 cells. The results show that direct manipulation of XBP-1 levels through knockdown of XBP-1 with shRNA did not have a marked effect on sensitivity of the cells to treatment with Bortezomib (FIG. 14C). This provides further evidence that XBP-1 is not itself the principle target of Bortezomib but can act as an indirect marker of dependence of the cell on the UPR.

### EXAMPLE 4

### Derivation of Bortezomib-Resistant Cell Lines

Myeloma cell lines KMS-11 and H292 were adapted to growth in the presence of Bortezomib. Independent pools of the cells were cultured (as indicated previously in Example 3.1.3) with continuous exposure to Bortezomib, beginning with a low concentration at ¼ IC50, and increasing gradually as the cells adapted. Cells were passaged and medium plus Bortezomib renewed at intervals of approximately one week. Over a period of months, the pools were eventually adapted to continuous exposure to 4 times the starting IC50. Total XBP-1 and sensitivity to Bortezomib was determined as previously described (Example 1.1 and Example 2.1, respectively).

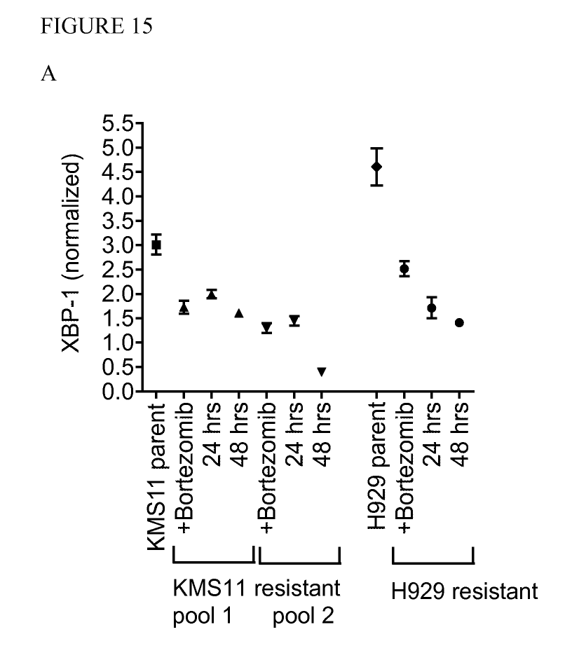

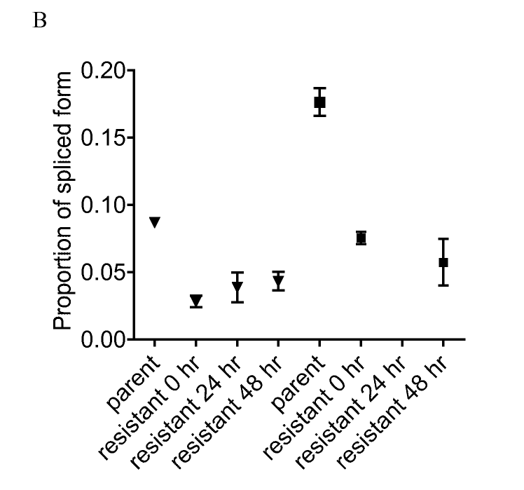

Bortezomib-resistant cell lines showed stable down-regulation of total XBP-1 mRNA levels (FIG. 15A). Moreover, the proportion of spliced (active) XBP-1 mRNA also decreased (FIG. 15B). This strongly supports the relationship between XBP-1 levels and Bortezomib sensitivity, and its use for predicting the response of myeloma patients to treatment with Bortezomib or other proteosome inhibitor.

### EXAMPLE 5

### Other Components of the Unfolded Protein Response (UPR) are Down-Regulated in Bortezomib-Resistant Cell Lines

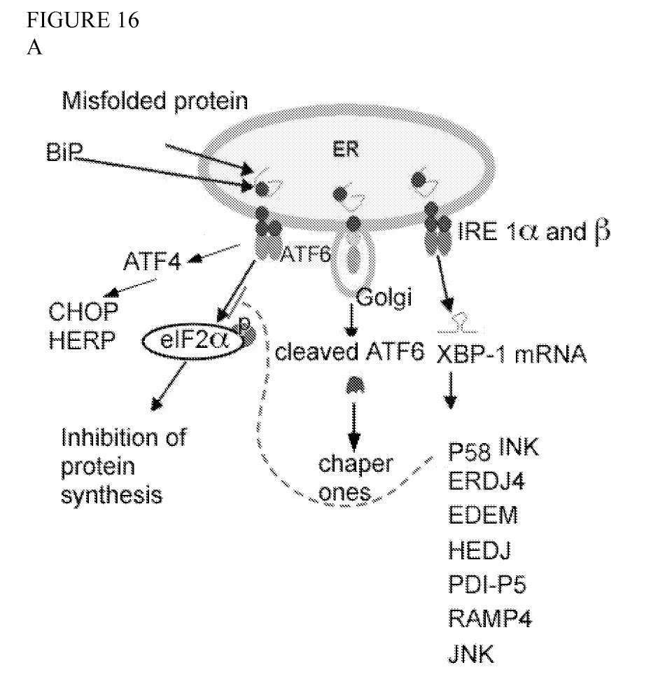

It was investigated whether in addition to XBP-1, Bortezomib-resistant myeloma cell lines exhibited down-regulation of other components of the UPR. FIG. 16A illustrates the various components of the UPR. Western Blot analysis was carried out to determine the protein levels in Bortezomib-resistant and corresponding non-resistant cell lines of the phosphorylated form of eIF2α, the chaperone BiP, the transcription factor ATF6 and the kinase inhibitor p58^{INK}.

5.1 SDS-PAGE and Western Blot Analysis

Bortezomib-resistant and non-resistant KMS-11 and H929 cell lines were resuspended in fresh medium (without Bortezomib) at approximately 10⁶ cells /mL. Two days later, the cells were counted (on a Beckmann-Coulter Z2 pore resistance counter) and harvested by centrifugation at 1500 g. The cells were washed in phosphate-buffered saline (PBS) and then lysed by resuspension at 4×10⁷/mL in 10 mM Tris HCl pH 8, 10 mM MgCl₂, 2 mM CaCl₂, 0.1% Triton X-100, plus Protease inhibitor cocktail (Sigma, as per manufacturer's instructions) followed by 3 freeze-thaw cycles. Nuclei and debris were pelleted by centrifugation at 18 000 g at 4° C. and the supernatant retained. Protein therein was quantified by Bradford assay. Lysates were mixed 2:1 with 3× Laemmli buffer, boiled 3 min and fractionated on 10% SDS-PAGE gels, 8 μg protein per lane. Gels were electroblotted onto PVDF membranes, blocked with 5% skim milk in PBST (phosphate buffered saline plus 0.1% (v/v) Tween-20) for 1 hr, incubated with monoclonal or polyclonal antibody for 1 hr, washed 3 times for 20 min each with PBST. Specifically-bound antibodies were detected with horseradish peroxidase (HRP)-coupled secondary antibodies and visualised by enhanced chemiluminescence on a Kodak 4000 MM image station. GAPDH was utilised as a loading control.

BiP and the phosphorylated form of eIF2α were detected with a rabbit monoclonal antibodies from Cell Signaling Technology (Boston, Mass.) cat. C50B12 and 119A11 respectively. ATF6 was detected with mouse monoclonal antibody 70B1413.1 from Imgenex (San Diego, Calif.) cat. IMG-273. P58INK (PRKRIR) was detected with rabbit polyclonal antibody from Bethyl Laboratories (Montgomery, Tex.), cat. A300-586A. GAPDH was detected with mouse monoclonal antibody 0411 from Santa Cruz Biotechnology (Santa Cruz, Calif.) cat. sc-47724. Horse-radish peroxidase conjugate of goat anti-mouse Ig secondary antibody was from Santa Cruz Biotechnology cat. sc-2005, and the and HRP-goat anti-rabbit Ig secondary antibody was from Upstate (www.upstate.com), cat. 12-348.

Both KMS-11 and H929 Bortezomib-resistant cell lines showed a down regulation in the levels of phosphorylated eIF2α, BiP, ATF6 and p58^{ink} (FIG. 16B). Thus, in addition to an observed downregulation of XBP-1, Bortezomib-resistant cell lines exhibited downregulation of other components of the UPR.

### EXAMPLE 6

### Myeloma Patients that do not Respond to Bortezomib have Myelomas that Express Low Levels of XBP-1

A study was performed to investigate the pre-Bortezomib treatment level of XBP-1 in the myelomas of patients that ultimately undergo Bortezomib treatment and emerge as non-responders. The patient response to Bortezomib was classified according to the European Group for Blood and Marrow Transplantation criteria (EBMT; www dot ebmt dot org).

6.1 Obtaining Myeloma Samples

Myeloma cells were obtained from 9 relapsed myeloma patients undergoing treatment at Royal Prince Alfred Hospital (RPAH), Sydney, Australia (Institute of Haematology, RPAH), who had not previously been treated with Bortezomib and were due for Bortezomib treatment. In addition, samples from a further 8 patients were obtained from the Australian Multicentre Bortezomib Induction and Reinduction (BIR) study (Principal investigators: Dr N Horvath and Dr L Bik). The myeloma cell samples were obtained pre-treatment with Bortezomib via marrow biopsy. Prior to cryopreservation of the biopsies, mononuclear cells (“buffy coat”) were obtained by centrifugation through a ficoll gradient. The mononuclear cells were cryopreserved until required by freezing in RPMI-1640 supplemented with 10% Foetal Bovine Serum and 10% dimethylsulfoxide (DMSO) as cryoprotectant, and stored in liquid nitrogen.

Myeloma cells were obtained from cryopreserved biopsies. The cryopreserved cells were thawed, diluted 10-fold with FACS buffer (phosphate-buffered saline containing 1% (v/v) foetal bovine serum), pelleted by centifugation at 1500 g and resuspended in 100 μL of the same. The cells were then stained for 30 min on ice with antibody-fluorochrome conjugates with CD38-PE (phycoerythrin) as a myeloma marker and CD14-APC (allophycocyanin) to mark non-myeloma mononuclear cells, in preparation for sorting by flow cytometry (FACS). Stained cells were washed once with 4 mL FACS buffer, pelleted by centrifugation at 1500 g and resuspended at approximately 10⁶/mL in FACS buffer containing 5 μM MgCl₂, 20 μg/mL DNAse I and 4 μg/mL DAPI, as a vital dye to mark non-viable cells. The gated population was CD38 high, CD14-ve, DAPI-ve.

To verify that the gating was appropriate, two small aliquots (5 μL each) of the unstained, unsorted cells were fixed in 4% formaldehyde for 15 min, washed twice in FACS buffer, permeabilised in 0.5% saponin in FACS buffer and stained in the same with fluorescein isothiocyanate (FITC) conjugates of polyclonal antibodies to kappa and lambda light chains. They were then stained with CD38-PE and CD14-APC as above but omitting DAPI. These were used to confirm that the gating yielded myeloma cells, where cytoplasmic light chain levels were highest. It also provided an approximate measure of immunoglobulin light chain levels in the myeloma samples.

The live, stained cells were then sorted (on a FACS Aria). Yields were in the range 10⁴-10⁶ myeloma cells.

6.2 Isolation of RNA from Purified Myeloma Cells

RNA was isolated from purified myeloma cells using the procedure as described in Example 1.1 with modification as follows.

Total RNA was extracted from cultured myeloma cells using Tri Reagent (isophasic guanidine isothiocyanate:phenol, MRC) according to manufacturer's protocol (TRI Reagent—RNA, DNA, protein isolation reagent. Manufacturer's protocol (1995), Molecular Research Center, Inc. Cincinnati, Ohio) Glycogen was added as carrier during precipitation of the RNA. The quality and approximate quantity of RNA was checked on BioAnalyzer 2100 pico RNA chips (Agilent Technologies; www dot agilent dot com) and then quantified by Ribogreen RNA Quanti assay (Invitrogen).

6.3 Generation of cDNA and Quantitation of Total Unspliced and Spliced XBP-1

cDNA was prepared as described previously in Example 1.1 with the following modifications.

First strand cDNA synthesis was generated with SuperScript™ III Reverse Transcriptase (Invitrogen), using from 0.1 to 1 μg of RNA, mixed Oligo dT and random hexamer primers according to the manufacturer's directions. Each RNA sample was reverse-transcribed twice to produce duplicate cDNA samples.

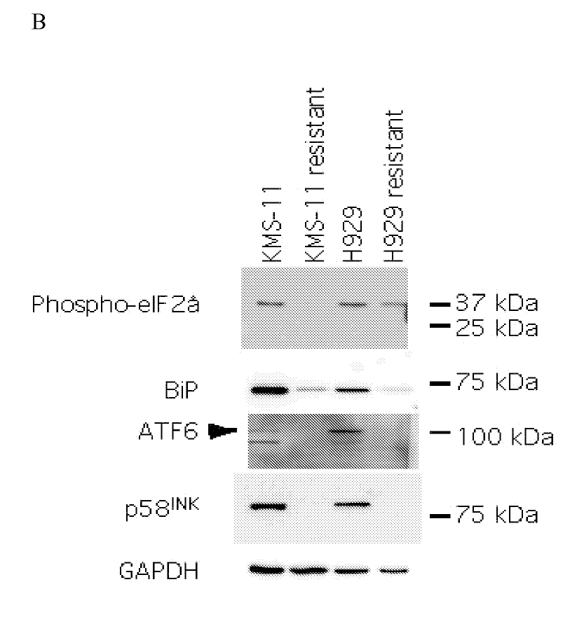

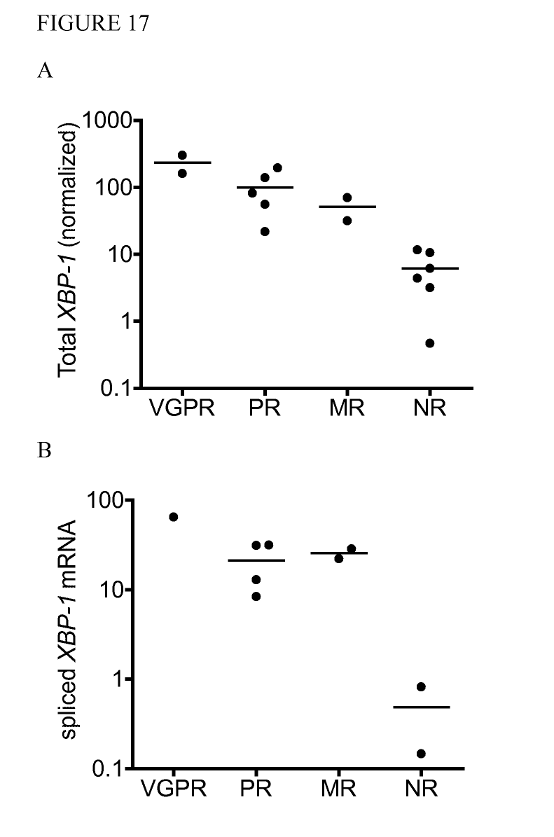

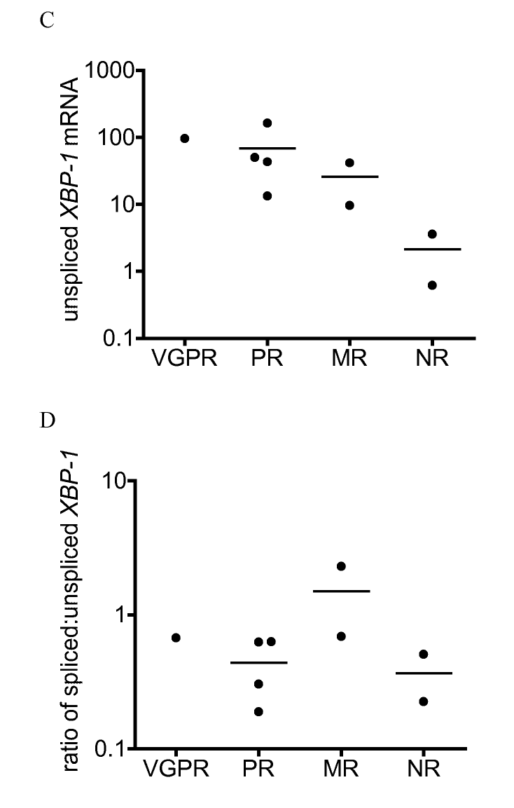

Real time PCR was performed as described in Example 1.1. Each cDNA sample was analyzed twice by real-time PCR to yield 4 data points per patient sample. The graphed data is the mean of the 4 datapoints for each patient (FIG. 17). The ratio of the spliced and unspliced forms of XBP-1 mRNA was determined by quantification of late log-phase XBP-1 PCR products on an Agilent BioAnalyzer and reference to standards consisting of known ratios of PCR products of the spliced and unspliced forms of the mRNA.

6.4 Results

FIG. 17 illustrates the relationship existing between pre-treatment levels of XBP-1 mRNA and the response of myeloma patients to Bortezomib. Patients that experienced either a minimal response (MR), partial response (PR) or a very good partial response (VGPR) to Bortezomib showed consistently higher XBP-1 mRNA levels than in patients who failed to respond (i.e., no reponse (NR)) to Bortezomib treatment (FIG. 17A). This relationship was also reflected in the measurement of unspliced XBP-1 mRNA levels (FIG. 17C) as well as spliced XBP-1 mRNA levels (FIG. 17B) in contrast with spliced XBP-1 mRNA levels measured in human myeloma cell lines which were less predictive of sensitivity to Bortezomib (FIGS. 8C and 9C). No clear relationship between the patients' response to Bortezomib and the ratio of spliced:unspliced XBP-1 mRNA was observed (FIG. 17D).

Two patients with high XBP-1 mRNA levels failed to respond to Bortezamib treatment. The 2 patients concerned were both atypical in that they deteriorated rapidly, and it is likely there was insufficient time for a significant response to the treatment to manifest.

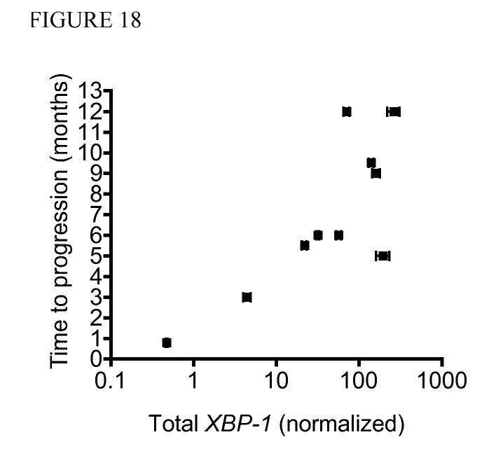

These results from myeloma marrow biopsies indicate that patients who fail to respond to Bortezomib have myeloma cells expressing the lowest levels of XBP-1 whereas patients who respond to Bortezomib have myeloma cells expressing higher levels of XBP-1. This is also reflected in the time to myeloma progression for patients treated with Bortezomib. Patients with a longer time to myeloma relapse were patients who responded well to Bortezomib and whose myeloma cells expressed higher levels of total XBP-1 mRNA (FIG. 18), whereas myeloma cells in patients relapsing soon after treatment expressed low levels of XBP-1 mRNA although relapse time was available for a subset of patients only.

While a number of embodiments have been described, it will be appreciated by persons skilled in the art that numerous variations and/or modifications may be made without departing from the spirit or scope of the invention as broadly described. The present embodiments are, therefore, to be considered in all respects as illustrative and not restrictive.

### REFERENCES

1. Richardson, P. G., Sonneveld, P., Schuster, M. W., Irwin, D., Stadtmauer, E. A., Facon, T., Harousseau, J. L., Ben-Yehuda, D., Lonial, S., Goldschmidt, H., Reece, D., San-Miguel, J. F., Blade, J., Boccadoro, M., Cavenagh, J., Dalton, W. S., Boral, A. L., Esseltine, D. L., Porter, J. B., Schenkein, D., and Anderson, K. C. Bortezomib or high-dose dexamethasone for relapsed multiple myeloma. N Engl J Med, 352: 2487-2498, 2005.

2. Jagannath, S., Barlogie, B., Berenson, J., Siegel, D., Irwin, D., Richardson, P. G., Niesvizky, R., Alexanian, R., Limentani, S. A., Alsina, M., Adams, J., Kauffinan, M., Esseltine, D. L., Schenkein, D. P., and Anderson, K. C. A phase 2 study of two doses of bortezomib in relapsed or refractory myeloma. Br J Haematol, 127: 165-172, 2004.

3. Richardson, P. G., Barlogie, B., Berenson, J., Singhal, S., Jagannath, S., Irwin, D., Rajkumar, S. V., Srkalovic, G., Alsina, M., Alexanian, R., Siegel, D., Orlowski, R. Z., Kuter, D., Limentani, S. A., Lee, S., Hideshima, T., Esseltine, D. L., Kauffman, M., Adams, J., Schenkein, D. P., and Anderson, K. C. A phase 2 study of bortezomib in relapsed, refractory myeloma. N Engl J Med, 348: 2609-2617, 2003.

4. Lee, K., Tirasophon, W., Shen, X., Michalak, M., Prywes, R., Okada, T., Yoshida, H., Mori, K., and Kaufman, R. J. IRE1-mediated unconventional mRNA splicing and S2P-mediated ATF6 cleavage merge to regulate XBP1 in signaling the unfolded protein response. Genes & Development, 16: 452-466, 2002.

5. Lee, A. H., Iwakoshi, N. N., and Glimcher, L. H. XBP-1 regulates a subset of endoplasmic reticulum resident chaperone genes in the unfolded protein response. Molecular & Cellular Biology, 23. 7448-7459, 2003.

6. Lee, A. H., Iwakoshi, N. N., Anderson, K. C., and Glimcher, L. H. Proteasome inhibitors disrupt the unfolded protein response in myeloma cells. Proceedings of the National Academy of Sciences of the United States of America, 100: 9946-9951, 2003.

7. Shi, Y., Vattem, K. M., Sood, R., An, J., Liang, J., Stramm, L., and Wek, R. C. Identification and characterization of pancreatic eukaryotic initiation factor 2 alpha-subunit kinase, PEK, involved in translational control. Molecular & Cellular Biology, 18: 7499-7509, 1998.

8. Gass, J. N., Gifford, N. M., and Brewer, J. W. Activation of an unfolded protein response during differentiation of antibody-secreting B cells. J Biol Chem, 277: 49047-49054, 2002.

9. Yoshida, H., Matsui, T., Yamamoto, A., Okada, T., and Mori, K. XBP1 mRNA is induced by ATF6 and spliced by IRE1 in response to ER stress to produce a highly active transcription factor. Cell, 107: 881-891, 2001.

10. Shaffer, A. L., Shapiro-Shelef, M., Iwakoshi, N. N., Lee, A. H., Qian, S. B., Zhao, H., Yu, X., Yang, L., Tan, B. K., Rosenwald, A., Hurt, E. M., Petroulakis, E., Sonenberg, N., Yewdell, J. W., Calame, K., Glimcher, L. H., and Staudt, L. M. XBP1, downstream of Blimp-1, expands the secretory apparatus and other organelles, and increases protein synthesis in plasma cell differentiation. Immunity, 21: 81-93, 2004.

11. Shaffer, A. L., Lin, K. I., Kuo, T. C., Yu, X., Hurt, E. M., Rosenwald, A., Giltnane, J. M., Yang, L., Zhao, H., Calame, K., and Staudt, L. M. Blimp-1 orchestrates plasma cell differentiation by extinguishing the mature B cell gene expression program. Immunity, 17: 51-62, 2002.

12. Reimold, A. M., Iwakoshi, N. N., Manis, J., Vallabhajosyula, P., Szomolanyi-Tsuda, E., Gravallese, E. M., Friend, D., Grusby, M. J., Alt, F., and Glimcher, L. H. Plasma cell differentiation requires the transcription factor XBP-1. [see comment]. Nature, 412: 300-307, 2001.

13. Iwakoshi, N. N., Lee, A. H., Vallabhajosyula, P., Otipoby, K. L., Rajewsky, K., and Glimcher, L. H. Plasma cell differentiation and the unfolded protein response intersect at the transcription factor XBP-1. Nature Immunology, 4: 321-329, 2003.

14. Nakamura, M., Gotoh, T., Okuno, Y., Tatetsu, H., Sonoki, T., Uneda, S., Mori, M., Mitsuya, H., and Hata, H. Activation of the endoplasmic reticulum stress pathway is associated with survival of myeloma cells. Leuk Lymphoma, 47: 531-539, 2006.

15. Hideshima, T., Chauhan, D., Richardson, P., Mitsiades, C., Mitsiades, N., Hayashi, T., Munshi, N., Dang, L., Castro, A., Palombella, V., Adams, J., and Anderson, K. C. NF-kappa B as a therapeutic target in multiple myeloma. Journal of Biological Chemistry, 277: 16639-16647, 2002.

16. Hideshima, T., Chauhan, D., Richardson, P., Mitsiades, C., Mitsiades, N., Hayashi, T., Munshi, N., Dang, L., Castro, A., Palombella, V., Adams, J., and Anderson, K. C. NF-kappa B as a therapeutic target in multiple myeloma. Journal of Biological Chemistry, 277: 16639-16647, 2002.

17. Michiels F, van der Kammen R A, Janssen L, Nolan G, Collard J G. Expression of Rho GTPases using retroviral vectors. Methods Enzymol 2000;325:295-302.

18. Grignani F, Kinsella T, Mencarelli A, Valtieri M, Riganelli D, Grignani F, Lanfrancone L, Peschle C, Nolan G P, Pelicci P G. High-efficiency gene transfer and selection of human hematopoietic progenitor cells with a hybrid EBV/retroviral vector expressing the green fluorescence protein. Cancer Research 1998; 58:14-19.

1. A method for predicting response to a proteasome inhibitor in the prophylaxis or treatment of a cancer in an individual, comprising:
providing a sample of cancer cells of the cancer from the individual;
evaluating the level of at least one molecule in the cancer cells associated with the unfolded protein response of the cancer cells, to provide test data indicative of the level of activity of the unfolded protein response; and
using the test data to predict the response of the cancer cells to the proteasome inhibitor.

2. A method according to claim 1 wherein the use of the test data comprises comparing the test data with reference data, the prediction being based on the comparison.

3. A method according to claim 1 wherein the at least one molecule is a protein or a nucleic acid encoding the protein.

4. A method according to claim 1 wherein evaluating the level of the at least one molecule comprises the steps of:
amplifying cDNA target nucleic acid encoding for the molecule utilising a process involving thermocycling and primers to obtain amplified product; and
measuring the amount of the amplified product.

5. A method according to claim 1 wherein the at least one molecule is a component of a signaling pathway of the unfolded protein response.

6. A method according to claim 5 wherein the signaling pathway is selected from the IRE1/XBP-1 and ATF-6 signaling pathways.

7. A method according to claim 1 wherein the at least one molecule is selected from the group consisting of XBP-1, ATF-6, BLIMP-1, DnaJ/Hsp40-like proteins, p58IPK, ERDj4, HEDJ, EDEM, protein disulfide isomerase-P5, ribosome-associated membrane protein 4 (RAMP4) and BiP.

8. A method according to claim 7 wherein the at least one molecule is XBP-1 protein or nucleic acid encoding XBP-1.

9. A method according to claim 8 wherein the at least one molecule is unspliced XBP-1 mRNA.

10. A method according to claim 8 wherein the at least one molecule is spliced XBP-1 protein or nucleic acid encoding spliced XBP-1.

11. A method according to claim 8 wherein the at least one molecule is total XBP-1 mRNA including unspliced and spliced XBP-1 mRNA.

12. A method according to claim 1 wherein the proteosome inhibitor is selected from the group consisting of Bortezomib, leupeptin, calpain inhibitor I, calpain inhibitor II, MG115, MG132, PSI, peptide glyoxal, peptide aldehyde, peptide benzamides, peptide α-ketoamides, peptide vinyl sulfones, peptide boronic acids, NLVS, PS-341, lactacystin, clasto-lactacystin β-lactone, PS-519, epoxomicin, eponemycin, TMC-86A, TMC-86B, TMC-89, TMC-96, YU 101, gliotoxin, HNE(4-hydroxy-2-nonenal), YU 102, NPI-0052 and PR-171.

13. A method according to claim 12 wherein the proteosome inhibitor is Bortezomib or MG132.

14. A method according to claim 1 wherein the cancer is a blood cell cancer.

15. A method according to claim 1 wherein the cancer is selected from the group consisting of myeloma, lymphoma, multiple myeloma, plasma cell leukemia and Waldenstrom macroglobulinemia.

16. A method for determining a treatment for cancer in an individual, comprising:
providing a sample of cancer cells of the cancer from the individual;
evaluating the level of at least one molecule in the cancer cells associated with the unfolded protein response of the cancer cells, to provide test data indicative of the level of activity of the unfolded protein response; and
using the test data to select a treatment for treating the cancer.

17. A method according to claim 16 wherein the use of the test data comprises comparing the test data with reference data, the prediction being based on the comparison.

18. A method according to claim 16 wherein the at least one molecule is a protein or a nucleic acid encoding the protein.

19. A method according to claim 16 wherein the at least one molecule is a component of a signaling pathway of the unfolded protein response.

20. A method according to claim 16 wherein the at least one molecule is selected from the group consisting of XBP-1, ATF-6, BLIMP-1, DnaJ/Hsp40-like proteins, p58IPK, ERDj4, HEDJ, EDEM, protein disulfide isomerase-P5, ribosome-associated membrane protein 4 (RAMP4) and BiP.

21. A method according to claim 16 wherein the at least one molecule is XBP-1 protein or nucleic acid encoding XBP-1.

22. A method according to claim 16 wherein the proteosome inhibitor is selected from the group consisting of Bortezomib, leupeptin, calpain inhibitor I, calpain inhibitor II, MG115, MG132, PSI, peptide glyoxal, peptide aldehyde, peptide benzamides, peptide α-ketoamides, peptide vinyl sulfones, peptide boronic acids, NLVS, PS-341, lactacystin, clasto-lactacystin β-lactone, PS-519, epoxomicin, eponemycin, TMC-86A, TMC-86B, TMC-89, TMC-96, YU 101, gliotoxin, HNE(4-hydroxy-2-nonenal), YU 102, NPI-0052 and PR-171.

23. A method according to claim 16 wherein the cancer is a blood cell cancer.


SEQUENCE LISTING (10 sequences)

SEQ ID NO: 1 - DNA, 74 bp, Homo sapiens
tctgctgagtccgcagcaggtgcaggcccagttgtcacccctccagaacatctccccatggattctggcggtat

SEQ ID NO: 2 - DNA, 26 bp, Homo sapiens
gcagcactcagactacgtgcacctct

SEQ ID NO: 3 - DNA, 20 bp, Homo sapiens
ggagttaagacagcgcttgg

SEQ ID NO: 4 - DNA, 19 bp, Homo sapiens
gtcaataccgccagaatcc

SEQ ID NO: 5 - DNA, 25 bp, Homo sapiens
accaactgggacgacatggagaaaa

SEQ ID NO: 6 - DNA, 20 bp, Homo sapiens
cgcacgatttcccgctcggc

SEQ ID NO: 7 - DNA, 22 bp, Homo sapiens
cggtgcctagtctggagctatg

SEQ ID NO: 8 - DNA, 31 bp, Homo sapiens
ccatcgatccttagacactaatcagctgggg

SEQ ID NO: 9 - DNA, 14 bp, Homo sapiens
ggcgctgggcggct

SEQ ID NO: 10 - DNA, 20 bp, Homo sapiens
acagagaaagggaggctggt